(19) United States
(12) Patent Application Publication
(10) Pub. No.: US 2021/0186541 A1
(43) Pub. Date: Jun. 24, 2021

(54) DEVICES AND METHODS FOR TREATING VASCULAR OCCLUSION

(71) Applicant: Inari Medical, Inc., Irvine, CA (US)

(72) Inventor: John Coleman Thress, Capistrano Beach, CA (US)

(21) Appl. No.: 17/125,397

(22) Filed: Dec. 17, 2020

Related U.S. Application Data
(60) Provisional application No. 62/949,967, filed on Dec. 18, 2019.

(51) Int. Cl. A61B 17/22

(52) U.S. Cl. CPC A61B 17/22031; A61B 2017/00867

(57) ABSTRACT

Systems and methods for the intravascular treatment of clot material within a blood vessel of a human patient are disclosed herein. In one embodiment, a system includes a coring element for coring and separating the clot material. The coring element can comprise a unitary structure having a first region, a second region, a third region, and a fourth region. The first region is adjacent to a proximal portion of the unitary structure and includes a first mouth configured to core and separate the clot material. The second region is distal of the first region, generally tubular, and includes a first plurality of interconnected struts. The third region is distal of the second region and includes a second mouth configured to core and separate the vascular thrombus. The fourth region is distal of the third region, generally tubular, and includes a second plurality of interconnected struts.

### CROSS-REFERENCE TO RELATED APPLICATION

This application claims the benefit of U.S. Provisional Patent Application No. 62/949,967, filed Dec. 18, 2019, and titled “DEVICES AND METHODS FOR TREATING VASCULAR OCCLUSION,” which is incorporated herein by reference in its entirety.

### TECHNICAL FIELD

The present technology generally relates to systems, methods, and devices for extracting thrombi from blood vessels of human patients. In particular, some embodiments of the present technology relate to systems for thrombus extraction from the peripheral vasculature of a human patient.

### BACKGROUND

Thrombosis is the local coagulation or clotting of the blood in a part of the circulatory system, and a thrombus is a blood clot formed in situ within the vascular system. A venous thrombus is a blood clot that forms within a vein. A common type of venous thrombosis is a deep vein thrombosis (DVT), which is the formation of a blood clot within a deep vein (e.g., predominantly in the legs). Nonspecific signs of a thrombosis may include pain, swelling, redness, warmness, and engorged superficial veins.

If the thrombus breaks off (embolizes) and flows towards the lungs, it can become a life-threatening pulmonary embolism (PE) (e.g., a blood clot in the lungs). In addition to the loss of life that can arise from PE, DVT can cause significant health issues such as post thrombotic syndrome, which can cause chronic swelling, pressure, pain, and ulcers due to valve and vessel damage. Further, DVT can result in significant health-care costs either directly or indirectly through the treatment of related complications and inability of patients to work.

Three processes are believed to result in venous thrombosis. First is a decreased blood flow rate (venous stasis), second is an increased tendency to clot (hypercoagulability), and the third is changes to the blood vessel wall. DVT formation typically begins inside the valves of the calf veins where the blood is relatively oxygen deprived, which activates certain biochemical pathways. Several medical conditions increase the risk for DVT, including diabetes, cancer, trauma, and antiphospholipid syndrome. Other risk factors include older age, surgery, immobilization (as with bed rest, orthopedic casts, and sitting on long flights), combined oral contraceptives, pregnancy, the postnatal period, and genetic factors. The rate of DVT increases dramatically from childhood to old age and, in adulthood, about 1 in 1,000 adults develop DVT annually.

Although current devices and methods of prevention and/or treatment of DVT exist, there are a number of shortcomings that have yet to be resolved, such as high incidence of DVT re-occurrence, use of devices not designed to remove large clot volumes, and/or complicated treatments involving multiple treatment devices and/or pharmaceuticals. Accordingly, new devices, systems, and methods of treating thrombus, and particularly DVT are desired.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a side view of a thrombectomy system configured in accordance with an embodiment of the present technology.

FIGS. 2A and 2B are side views of a thrombus extraction assembly of the thrombectomy system including a thrombus extraction device in a partially-expanded configuration and a fully-expanded configuration, respectively, configured in accordance with embodiments of the present technology.

FIGS. 3A-3D are an isometric view, a side view, a top view, and a rear view, respectively, of a coring element of the thrombus extraction device configured in accordance with embodiments of the present technology.

FIG. 4 is an enlarged side view of the thrombus extraction device coupled to a distal portion of the thrombus extraction assembly and in the fully-expanded configuration in accordance with an embodiment of the present technology.

FIGS. 5A and 5B are side views of a dilator assembly of the thrombectomy system in a first configuration and a second configuration, respectively, configured in accordance with embodiments of the present technology.

FIG. 6 is an enlarged cross-sectional side view of a portion of the thrombectomy system including a self-expanding funnel configured in accordance with an embodiment of the present technology.

FIGS. 7A-7D are side views of the dilator assembly positioned within an introducer assembly of the thrombectomy system and illustrating various stages in a process or method for deploying the self-expanding funnel in accordance with embodiments of the present technology.

FIGS. 8A, 8C, and 8D are cross-sectional side views, and FIG. 8B is an enlarged cross-sectional isometric view, of a control assembly of the dilator assembly configured in accordance with embodiments of the present technology.

FIGS. 9A-9C, are cross-sectional side views of a control assembly configured in accordance with another embodiment of the present technology.

FIGS. 10A and 10B are partially cross-sectional side views of a control assembly configured in accordance with another embodiment of the present technology.

FIG. 11 is a schematic view of an introduction technique for accessing a thrombus for treatment with the thrombectomy system in accordance with an embodiment of the present technology.

FIGS. 12A-12C are side views, and FIGS. 12D-12K are enlarged side views, of the thrombectomy system positioned within a blood vessel during a thrombectomy procedure in accordance with embodiments of the present technology.

### DETAILED DESCRIPTION

The present technology is generally directed to methods and systems for removing clot material (e.g., a thrombus) from a blood vessel of a human patient. In some embodiments, a system for removing clot material (e.g., a thrombectomy system) includes a thrombus extraction device including (i) a coring element configured to core and separate the clot material from the vessel wall and (ii) a capture element configured to capture the cored and separated clot material. In some embodiments, the coring element comprises a unitary structure having a first region adjacent to a proximal portion of the unitary structure, a second region distal of the first region, a third region distal of the second region, and a fourth region distal of the third region. The first region can include a first mouth configured to core and separate the clot material and the third region can include a second mouth configured to core and separate the clot material. The second and fourth regions can each be generally tubular and can include a plurality of interconnected struts. In one aspect of the present technology, the first and second mouths are radially offset such that at least one of the first and second mouths is positioned and oriented to effectively core and separate the clot material from within the blood vessel during a thrombus extraction procedure using the thrombus extraction device.

In some embodiments, the thrombectomy system includes a dilator assembly for deploying an expandable funnel coupled to a distal portion of an introducer sheath. The dilator assembly can include a first shaft defining a lumen, a second shaft slidably positioned within the lumen of the first shaft, and a retention sheath coupled to the second shaft and configured to receive and constrain the funnel therein. A control assembly including an actuator is operably coupled to the first and second shafts. Movement of the actuator to a first position is configured to distally advance the first and second shafts together to deploy the funnel from the retention sheath. Movement of the actuator to a second position is configured to distally advance the first shaft relative to the second shaft such that first shaft and the retention sheath define a generally uniform (e.g., constant diameter) outer surface. In one aspect of the present technology, the generally uniform outer surface of the dilator assembly is unlikely to snag or otherwise damage the funnel or vessel as the dilator assembly is retracted through the introducer sheath. In another aspect of the present technology, the dilator assembly can be coupled to the introducer sheath to inhibit or even prevent unintentional, premature deployment of the funnel.

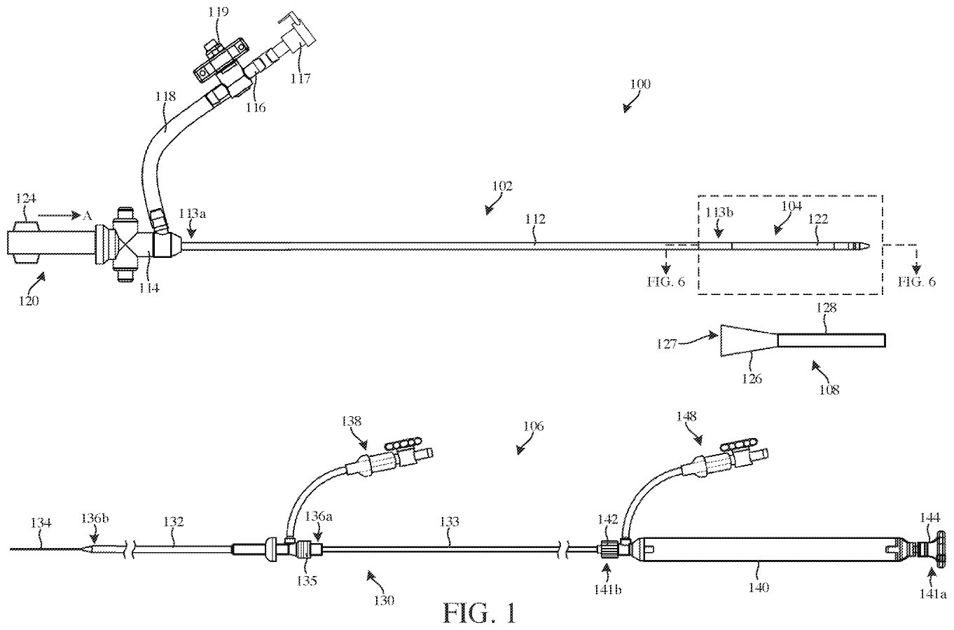

Although many of the embodiments are described below with respect to devices, systems, and methods for treating vascular thrombi (e.g., deep vein thrombosis (DVT)), other applications and other embodiments in addition to those described herein are within the scope of the technology (e.g., intravascular procedures other than the treatment of emboli, intravascular procedures for treating cerebral embolism, intravascular procedures for treating pulmonary embolism). In general, for example, the devices, systems, and methods of the present technology can be used to extract any formation of material in a vessel (e.g., a venous or arterial vessel), such as cancerous growths, vegetation, and the like. Additionally, several other embodiments of the technology can have different configurations, states, components, or procedures than those described herein. Moreover, it will be appreciated that specific elements, substructures, advantages, uses, and/or other features of the embodiments described with reference to FIGS. 1-12K can be suitably interchanged, substituted or otherwise configured with one another in accordance with additional embodiments of the present technology. Furthermore, suitable elements of the embodiments described with reference to FIGS. 1-12K can be used as standalone and/or self-contained devices. A person of ordinary skill in the art, therefore, will accordingly understand that the technology can have other embodiments with additional elements, or the technology can have other embodiments without several of the features shown and described below with reference to FIGS. 1-12K.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to use in a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### I. SELECTED EMBODIMENTS OF THROMBECTOMY SYSTEMS

FIG. 1 is a side view of a thrombectomy system 100 (which can also be referred to as a thrombus extraction system, clot removal system) configured in accordance with an embodiment of the present technology. In the illustrated embodiment, the thrombectomy system 100 includes an introducer assembly 102, an obturator or dilator assembly 104 (shown positioned within the introducer assembly 102), and a thrombus extraction assembly 106. In general, the thrombectomy system 100 can be used to (i) access a portion of a blood vessel (e.g., a venous vessel of a human patient) containing a thrombus (e.g., clot material) and (ii) remove all or portions of that thrombus from the blood vessel. More specifically, for example, the introducer assembly 102 and the dilator assembly 104 can be partially advanced into the vasculature of the patient (e.g., a blood vessel or venous vessel of the patient). The dilator assembly 104 can be actuated to deploy a self-expanding funnel (e.g., as shown in FIGS. 7A-7C) and then removed from the introducer assembly 102. Next, the thrombus extraction assembly 106 and an attached thrombus extraction device can be partially inserted through the introducer assembly 102 and deployed at and/or near the location of a thrombus for capturing the thrombus. Finally, the thrombus extraction assembly 106 and/or the introducer assembly 102 can be removed from the patient along with the captured thrombus. In some embodiments, the thrombectomy system 100 and/or methods of operating the thrombectomy system 100 to remove a thrombus from a patient can include some features the same as or similar to the thrombectomy systems described in detail in (i) U.S. Pat. No. 9,700,332, filed Sep. 16, 2016, and titled “INTRAVASCULAR TREATMENT OF VASCULAR OCCLUSION AND ASSOCIATED DEVICES, SYSTEMS, AND METHODS,” and/or (ii) U.S. Pat. No. 10,098,651, filed Apr. 26, 2017, and titled “DEVICES AND METHODS FOR TREATING VASCULAR OCCLUSION,” both of which are incorporated herein by reference in their entirety.

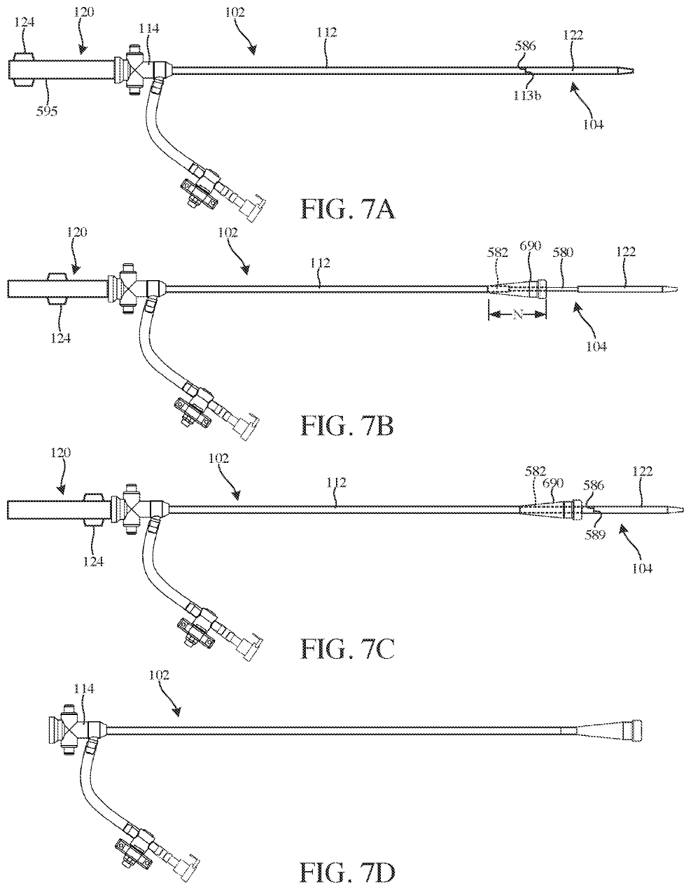

In the illustrated embodiment, the introducer assembly 102 includes an elongate sheath 112, which can also be referred to as a shaft, catheter, and the like. The sheath 112 defines a lumen (obscured in FIG. 1; e.g., identified as lumen 688 in FIG. 6) and includes a proximal portion 113a and a distal portion 113b. The proximal portion 113a can terminate at a proximal end, and the distal portion 113b can terminate at a distal end. The lumen of the sheath 112 is sized to slidably receive the dilator assembly 104 and the thrombus extraction assembly 106. For example, the dilator assembly 104 is shown partially positioned within the sheath 112 in FIG. 1. The sheath 112 can be elastic and/or flexible and can have any suitable length and diameter. In some embodiments, the sheath 112 can have an outer diameter of at least 10 French, at least 12 French, at least 14 French, at least 18 French, at least 20 French, at least 22 French, at least 26 French, greater than 26 French, between 10 French and 26 French, between 14 French and 24 French, between 15 French and 21 French, between 16 French and 22 French, and/or any other or intermediate size. In some embodiments, the lumen of the sheath 112 can have an internal diameter of at least 2 French, at least 10 French, at least 14 French, at least 18 French, at least 20 French, at least 22 French, between 11 French and 12 French, between 10 French and 22 French, between 14 French and 21 French, between 16 French and 20 French, and/or any other or intermediate size. In some embodiments, the sheath 112 can include a radiopaque marker (not shown) positioned, for example, at the distal portion 113b thereof.

The introducer assembly 102 further includes a sealable hub 114 coupled to the proximal portion 113a of the sheath 112. The sealable hub 114 is configured to allow access to the lumen of the sheath 112 and can be self-sealing and/or can comprise a self-sealing seal. For example, in the illustrated embodiment the sealable hub 114 is a hemostasis valve that is configured to maintain hemostasis during a thrombus extraction procedure by preventing fluid flow in the proximal direction through the sealable hub 114 as various components—such as portions of the dilator assembly 104 and/or the thrombus extraction assembly 106—are inserted through the sealable hub 114 to be delivered through the sheath 112 to a treatment site in a blood vessel. More specifically, the sealable hub 114 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety. The sealable hub 114 can include one or more buttons or actuators that enable an operator to selectively seal/unseal the sealable hub 114.

The introducer assembly 102 can further include an aspiration port 116 connected to the sealable hub 114 (e.g., to a side port of the sealable hub 114) and/or the sheath 112 (e.g., to the proximal portion 113a of the sheath 112) via, for example, a connecting tube 118. The aspiration port 116 can be connected to a syringe connector 117 that can be selectively coupled to a syringe or other aspiration device, or the aspiration port 116 can be connected to other suitable elements. In some embodiments, the introducer assembly 102 includes a fluid control device 119 configured to selectively fluidly connect the aspiration port 116 to the lumen of the sheath 112. In the illustrated embodiment, the fluid control device 119 is a stopcock operably coupled to the connecting tube 118 between the lumen of the sheath 112 and the aspiration port 116. In other embodiments, the fluid control device 119 can be a clamp or another suitable valve.

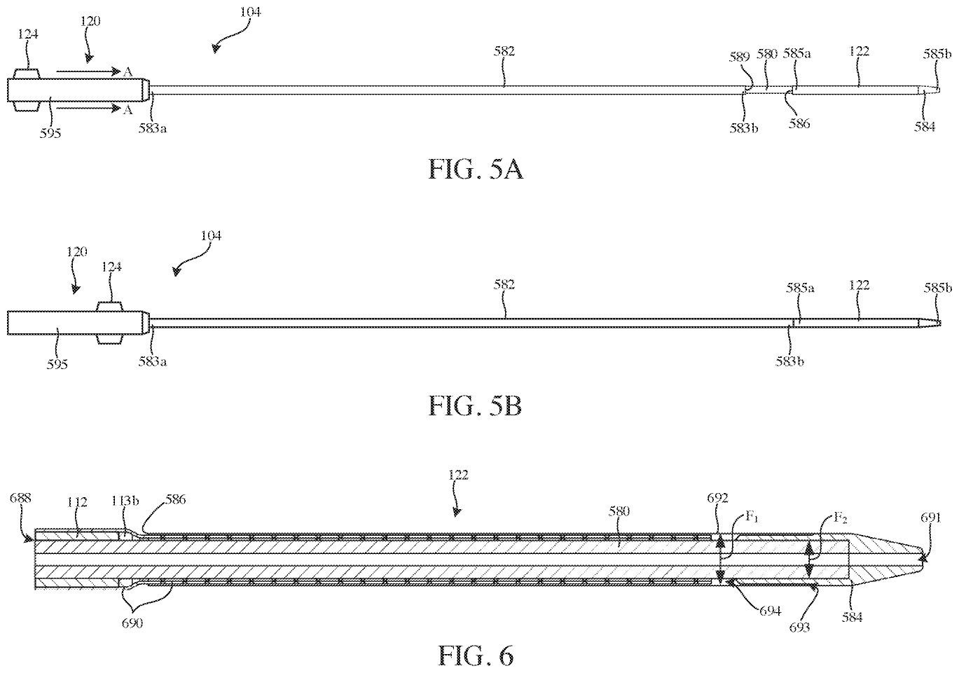

The dilator assembly 104 can include a control assembly 120 operably coupled to a retention sheath 122 via a first shaft (obscured in FIG. 1; e.g., identified as first shaft 580 in FIGS. 5A and 5B). In the illustrated embodiment, the first shaft of the dilator assembly 104 extends through the sealable hub 114 and the sheath 112 such that the retention sheath 122 is positioned distal of the distal portion 113b of the sheath 112. Moreover, the control assembly 120 is releasably coupled to (e.g., mated to, fixed to) the sealable hub 114. Accordingly, the introducer assembly 102 can carry or hold the dilator assembly 104. As described in greater detail below with reference to FIGS. 5A-7D, the dilator assembly 104 (e.g., the retention sheath 122) is configured to (i) hold/constrain a self-expanding funnel (obscured in FIG. 1; e.g., identified as funnel 690 in FIG. 6) that is attached to the distal portion 113b of the sheath 112, and (ii) release/deploy the self-expanding funnel. More specifically, for example, the control assembly 120 can include an actuator 124 that is movable (e.g., in the direction of arrow A in FIG. 1) to advance the retention sheath 122 relative to the sheath 112 (and the self-expanding funnel) attached thereto to deploy/release the self-expanding funnel.

In some embodiments, the thrombectomy system 100 can further include a loading tool 108 (e.g., a loading funnel) for use in loading the self-expanding funnel into the dilator assembly 104 (e.g., into the retention sheath 122). In the illustrated embodiment, the loading tool 108 defines a lumen 127 therethrough and includes a first portion 126 of varying diameter (e.g., a tapered portion such as a funnel portion) and a second portion 128 of generally constant diameter (e.g., a shaft portion). In other embodiments, the second portion 128 can have a partially varying diameter. The first portion 126 is configured (e.g., sized and shaped) to receive the self-expanding funnel and to move the self-expanding funnel to the constrained configuration as the self-expanding funnel is advanced through the first portion 126. The lumen 127 of the loading tool 108 can be sized to allow the retention sheath 122 to pass completely through the loading tool 108.

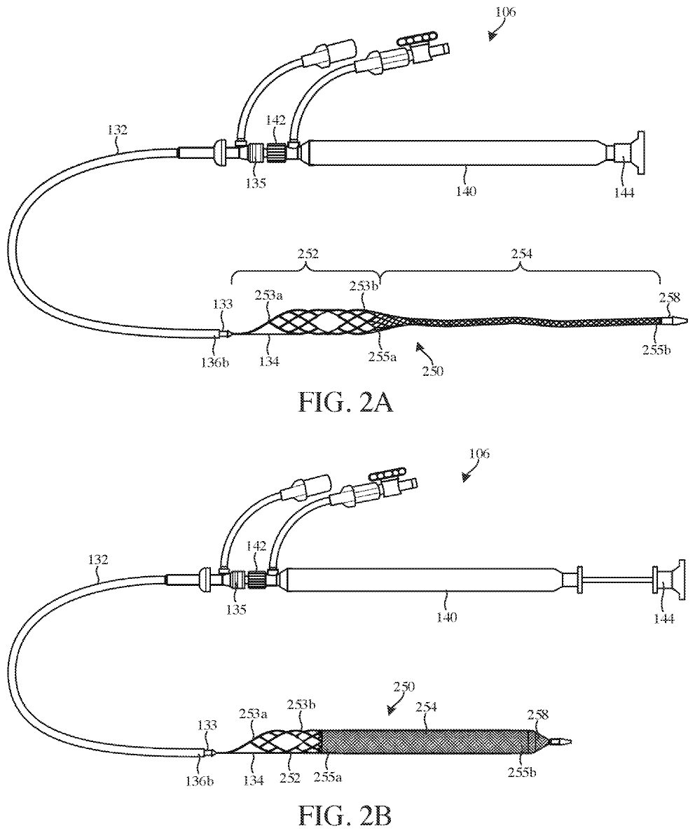

In the illustrated embodiment, the thrombus extraction assembly 106 includes a catheter portion 130 and a handle portion 140 (“handle 140”) operably coupled to the catheter portion 130. In operation, the handle 140 is configured to be actuated/manipulated by a user to control (e.g., deploy) one or more components of the catheter portion 130 and/or a thrombus extraction device (not shown in FIG. 1; e.g., identified as thrombus extraction device 250 in FIGS. 2A and 2B) coupled to the catheter portion 130.

In the illustrated embodiment the catheter portion 130 includes an outer shaft 132, an intermediate shaft 133, and an inner shaft 134 slidably and coaxially aligned relative to one another. For example, each of the shafts 132-134 can define a lumen (e.g., a central, axial lumen) and (i) the intermediate shaft 133 can be configured (e.g., sized and shaped) to slidably fit within the lumen of the outer shaft 132 and (ii) the inner shaft 134 can be configured to slidably fit within the lumen of the intermediate shaft 133. In some embodiments, the outer shaft 132 is configured (e.g., sized) to slidably fit within the sheath 112 of the introducer assembly 102 and can have, for example, a size of at least 8 French, at least 10 French, at least 11 French, at least 12 French, at least 14 French, at least 16 French, between 8 French and 14 French, between 11 French and 12 French, and/or any other or intermediate size. By this arrangement, each of the shafts 132-134 can be displaced longitudinally relative to one another and relative to the sheath 112 of the introducer assembly 102. In some embodiments, each of the shafts 132-134 can have the same length while, in other embodiments, one or more of the shafts 132-134 can have different lengths. For example, in some embodiments the intermediate shaft 133 can be longer than the outer shaft 132 and the inner shaft 134 can be longer than the intermediate shaft 133. In other embodiments, the catheter portion 130 can comprise any number of shafts (e.g., catheters, sheaths) that are slidable relative to one another and/or configured to be positioned coaxially relative to one another. For example, in some embodiments the catheter portion can include three intermediate shafts as described in detail in U.S. Pat. No. 10,098,651, filed Apr. 26, 2017, and titled “DEVICES AND METHODS FOR TREATING VASCULAR OCCLUSION,” which is incorporated herein by reference in its entirety.

The handle 140 includes a proximal portion 141a (e.g., a plunger portion) and a distal portion 141b (e.g., a locking portion). In the illustrated embodiment, the intermediate shaft 133 is coupled to and extends distally from the distal portion 141b of the handle 140. The distal portion 141b of the handle 140 can include a lock feature 142 such as, for example, a spinlock. The lock feature 142 is configured to selectively engage and/or lockingly engage with a mating feature 135 located near a proximal portion 136a of the outer shaft 132. In some embodiments, the outer shaft 132 can slide proximally over the intermediate shaft 133 until the lock feature 142 engages with the mating feature 135 to thereby secure the position of the outer shaft 132 relative to the intermediate shaft 133. In some embodiments, the intermediate shaft 133 is relatively longer than the outer shaft 132 such that a portion of the intermediate shaft 133 extends distally from a distal portion 136b of the outer shaft 132 when the outer shaft 132 is lockingly engaged with the lock feature 142.

In the illustrated embodiment, the handle 140 further includes a plunger 144 (e.g., an actuator) operably coupled to the inner shaft 134 and movable between a first, non-extended position (e.g., as shown in FIGS. 1 and 2A) and a second, extended position (e.g., as shown in FIG. 2B). Thus, movement of the plunger 144 relative to the handle 140 displaces the inner shaft 134 relative to the handle 140, the outer shaft 132, and/or the intermediate shaft 133. For example, withdrawing the plunger 144 proximally from the first position to the second position can withdraw the inner shaft 134 through the intermediate shaft 133. In some embodiments, the inner shaft 134 can have a length such that the inner shaft 134 extends distally past a distal terminus of the intermediate shaft 133 when the plunger 144 is in both the first and second positions. In some embodiments, the plunger 144 can be lockable in the first position and/or the second position to lock the position of the inner shaft 134. In other embodiments, the plunger 144 can be operably coupled to other components of the catheter portion 130 such as, for example, the intermediate shaft 133 and/or one or more additional shafts (not shown).

In the illustrated embodiment, the thrombus extraction assembly 106 further includes a first flush port 138 connected to the outer shaft 132 and a second flush port 148 connected to the handle 140. The first flush port 138 can be fluidly connected to the lumen of the outer shaft 132 to allow flushing of the lumen of the outer shaft 132. The second flush port 148 can be fluidly connected to the lumen of the intermediate shaft 133 (e.g., via an internal portion of the handle 140) to allow flushing of the lumen of the intermediate shaft 133.

The thrombus extraction assembly 106 can include and/or be coupled to a thrombus extraction device configured to core and capture a thrombus from the patient. FIGS. 2A and 2B, for example, are side views of the thrombus extraction assembly 106 of FIG. 1 operably coupled to a thrombus extraction device 250 configured in accordance with embodiments of the present technology. The thrombus extraction device 250 is shown in a deployed and partially-expanded configuration in FIG. 2A and a deployed and fully-expanded configuration in FIG. 2B. The thrombus extraction device 250 can be in an undeployed, constrained (e.g., unexpanded) position when positioned within the outer shaft 132.

Referring to FIGS. 2A and 2B together, the thrombus extraction device 250 includes an expandable coring element 252 and an expandable capture element 254 coupled to (e.g., attached to, connected to, integrally formed with) the coring element 252. The coring element 252 is positioned proximal of the capture element 254. In the illustrated embodiment, the coring element 252 includes (i) a proximal portion 253a coupled to the intermediate shaft 133 (e.g., to a distal portion of the intermediate shaft 133) and (ii) a distal portion 253b coupled to a proximal portion 255a of the capture element 254. Further, a distal portion 255b of the capture element 254 is coupled to the inner shaft 134 (e.g., to a distal portion of the inner shaft 134). As shown, the outer shaft 132 is proximally displaced relative to the handle 140 such that the mating feature 135 of the outer shaft 132 contacts/engages the lock feature 142 of the handle 140. Due to this positioning of the outer shaft 132 relative to the handle 140, each of the intermediate shaft 133, the inner shaft 134, and the thrombus extraction device 250 extend distally beyond the distal portion 136b of the outer shaft 132.

In some embodiments, the thrombus extraction device 250 can further include an atraumatic tip 258. In some embodiments, the atraumatic tip 258 can include a radiopaque marker to aid in intravascularly positioning the thrombus extraction device 250 within the patient. The thrombus extraction device 250 can additionally or alternatively include one or more radiopaque markers located on, for example, the outer shaft 132 (e.g., the distal portion 136b of the outer shaft 132) the intermediate shaft 133 (e.g., the distal portion of the intermediate shaft 133), and or other components of the thrombus extraction device 250. In some embodiments, the atraumatic tip 258 can define a channel configured to receive a guidewire therethrough.

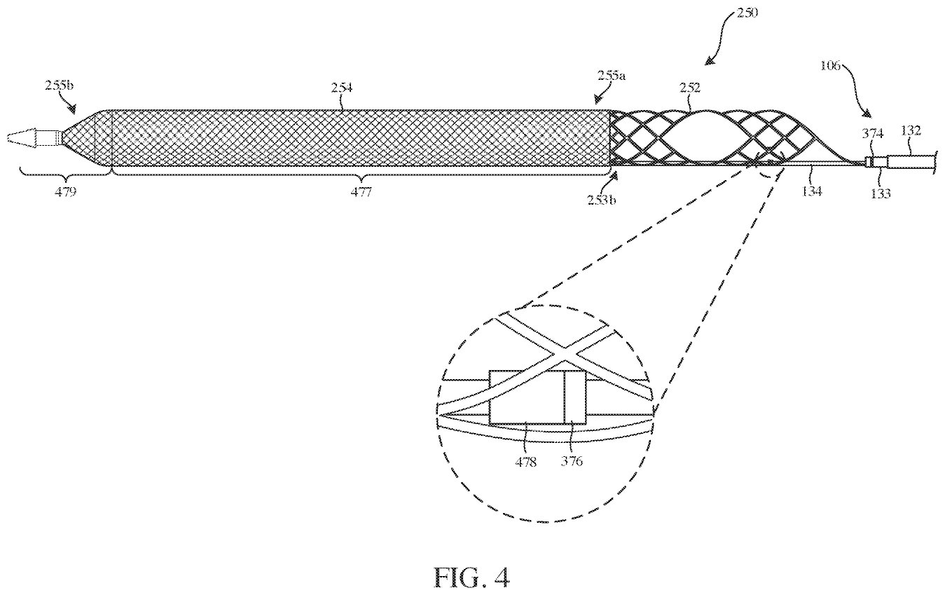

In the partially-expanded configuration shown in FIG. 2A, the plunger 144 of the handle 140 is in the first position. In contrast, in the fully-expanded configuration shown in FIG. 2B, the plunger 144 is in the second position (e.g., proximally retracted away from the handle 140) such that the inner shaft 134 is proximally retracted relative to the intermediate shaft 133. This proximal retraction of the inner shaft 134 relative to the intermediate shaft 133 forces the coring element and capture element 254 to fully expand, as described in greater detail below with reference to FIG. 4.

The thrombus extraction assembly 106 can comprise one or several features configured to secure the thrombus extraction device 250, and specifically the coring element 252 and/or the expandable capture element 254 in the fully-expanded position. As used herein, full expansion describes a condition in which the thrombus extraction device 250 is continually biased toward expansion by one or several forces in addition to the self-expanding forces arising from the thrombus extraction device 250. In some embodiments, full expansion occurs when the thrombus extraction device 250 is deployed and when the plunger 144 is in the second position (e.g., when the inner shaft 134 is proximally retracted relative to the intermediate shaft 133). Alternatively or additionally, full-expansion can occur when the thrombus extraction device 250 is deployed and biased towards expansion via a spring connected either directly or indirectly to the thrombus extraction device 250. Accordingly, when the thrombus extraction device 250 is fully expanded, forces less than a minimal radial compressive force do not change the diameter of the thrombus extraction device 250. Therefore, when fully-expanded, the thrombus extraction device 250 can maintain at least a desired radial force on a blood vessel when the thrombus extraction device 250 is drawn through that blood vessel. In some embodiments, the dimensions of the thrombus extraction device 250 can be selected such that the thrombus extraction device 250 apposes a wall of the blood vessel and/or applies a desired force to the wall of the blood vessel when fully expanded.

In some embodiments, the plunger 144 can be locked in the second position by, for example, rotating the plunger 144 with respect to the handle 140 to thereby engage one or several locking features on the plunger 144 and/or in the handle 140. Locking the plunger 144 in the second position secures the position of the inner shaft 134 relative to the intermediate shaft 133, thereby securing the thrombus extraction device 250 in the fully-expanded position. In other embodiments, the inner shaft 134 and the intermediate shaft 133 can be directly locked together via for example, (i) a static coupling in which the position of the inner shaft 134 is fixed relative to the position of the intermediate shaft 133 or (ii) a dynamic coupling in which the position of the inner shaft 134 relative to the intermediate shaft 133 is limited (rather than fixed). For example, the inner shaft 134 can be dynamically locked to the plunger 144 via a compliance spring (e.g., a tension spring, compression spring), which allows limited movement of the inner shaft 134 relative to the intermediate shaft 133 when the plunger 144 is locked in the second position.

### II. SELECTED EMBODIMENTS OF CORING ELEMENTS

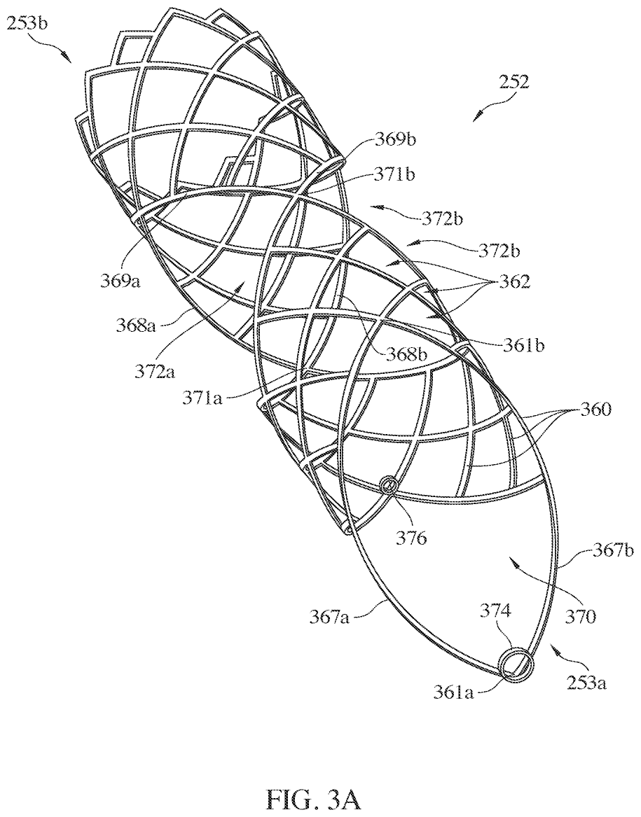

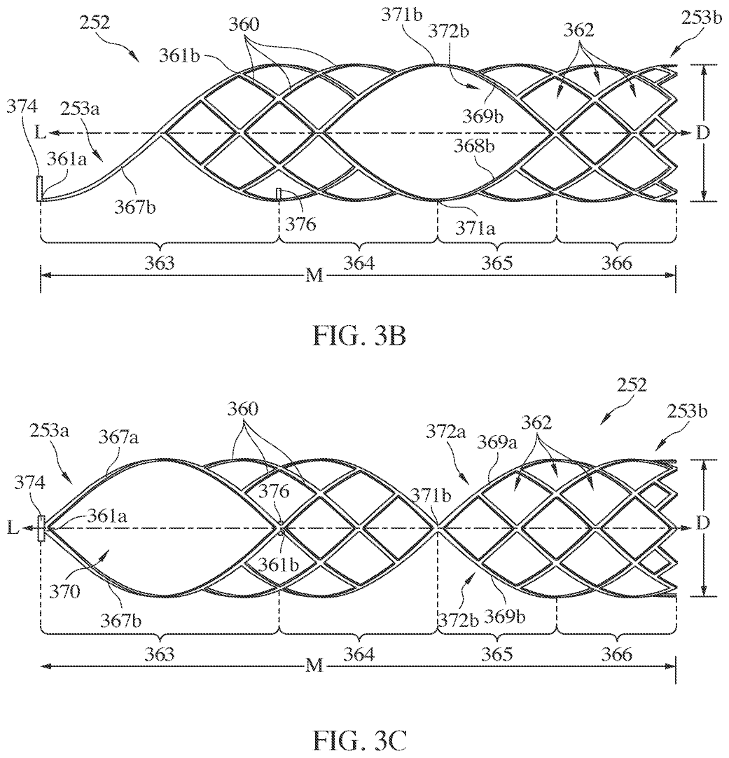

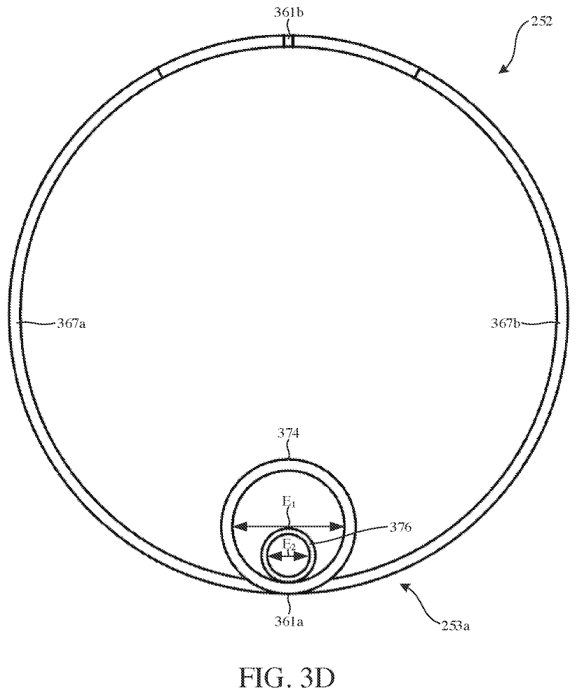

FIGS. 3A-3D are an isometric view, a side view, a top view, and a (proximally-facing) rear view, respectively, of the coring element 252 of the thrombus extraction device 250 of FIGS. 2A and 2B configured in accordance with embodiments of the present technology. Referring to FIGS. 3A-3D together, the coring element 252 comprises a plurality of struts 360 that together define a plurality of interstices or pores 362. The struts 360 can have a variety of shapes and sizes and, in some embodiments, the struts 360 can have a thickness and/or diameter between about 0.05-0.15 inch, between about 0.075-0.125 inch, between about 0.09-0.1 inch, about 0.096 inch, and/or other dimensions. In general, the struts 360 can together form a unitary fenestrated structure that is configured to core and separate a portion of a thrombus (e.g., a vascular thrombus) from a blood vessel containing the thrombus. In some embodiments, the coring element 252 can comprise a stent or stent-like device.

As best shown in FIGS. 3B and 3C, the coring element 252 includes a first region 363 including the proximal portion 253a, a second region 364 distal of the first region 363, a third region 365 distal of the second region 364, and a fourth region 366 distal of the third region 365 and including the distal portion 253b. The second region 364 and the fourth region 366 can be generally tubular. The first region 363 and the third region 365 have relatively fewer of the struts 360 compared to the second region 364 and the fourth region 366. For example, the first region 363 can include a pair of curved struts 367 (identified individually as first strut 367a and second strut 367a as best shown in FIGS. 3A and 3C) that curve in opposite directions around a central axis L of the coring element 252 and intersect and/or terminate at a pair of first junctions 361 (identified individually as a lower first junction 361a and an upper first junction 361b) to define a proximal, first mouth 370. The third region 365 can include (i) a pair of curved lower struts 368 (identified individually as a first lower strut 368a and a second lower strut 368b shown together in FIG. 3A) that extend distally from a lower second junction 371a and curve around the central axis L and (ii) a pair of curved upper struts 369 (identified individually as a first upper strut 369a and a second upper strut 369b shown together in FIGS. 3A and 3C) that extend distally from an upper second junction 371b and curve around the central axis L. The lower and upper struts 368, 369 together define a distal first mouth portion 372a and a distal second mouth portion 372b (collectively “a second mouth 372”). In the illustrated embodiment, the first mouth portion 372a is rotationally offset from the second mouth portion 372b. In other embodiments, the first mouth portion 372a can be positioned differently relative to the second mouth portion 372b (e.g., in a different rotational and/or longitudinal direction) and/or the second mouth 372 can comprise more than two separate portions (e.g., three, four, or more openings). In general, the first and second mouths 370, 372 can be defined by/in regions of the coring element 252 having different porosities.

In some embodiments, the coring element 252 is made from a shape memory material such as a shape memory alloy and/or a shape memory polymer. For example, the coring element 252 can comprise nitinol and/or a nitinol alloy. Similarly, the coring element 252 can be made using a variety of techniques including welding, laser welding, cutting, laser cutting, and/or expanding. For example, the coring element 252 can first be laser cut from a piece of nitinol (e.g., a nitinol tube) and then blown up and/or expanded. In general, the size (e.g., the length and diameter) of the coring element 252 can be selected based on the size (e.g., diameter) of the blood vessel from which thrombus is to be extracted. In some embodiments, the coring element 252 can have a length M of between about 0.2-5 inches (e.g., between about 1.5-2.5 inches, between about 1.75-2.25 inches, between about 1.9-2.0 inches, between about 1.5-1.8 inches, about 1.6 inches, about 1.7 inches, about 1.96 inches, about 3.0 inches, about 4.0 inches, smaller than 0.5 inch). In some embodiments, in the fully-expanded position unconstrained within a vessel, the coring element 252 can have a diameter D of between about 2-50 mm (e.g., between about 4-25 mm, between about 6 20 mm, between about 8-16 mm). In some embodiments, the length M of the coring element 252 can be selected based on the fully expanded and unconstrained diameter D of the coring element 252 to prevent undesired tipping and/or rotation of the coring element 252 within the blood vessel during operation. In general, the length M and the unconstrained diameter D of the coring element 252 will vary depending on the size of the vessel the coring element 252 is designed for. For example, the coring element 252 will generally have a smaller length M and diameter D when designed for smaller (e.g., 4 mm) vessels rather than larger (e.g., 25-35 mm) vessels.

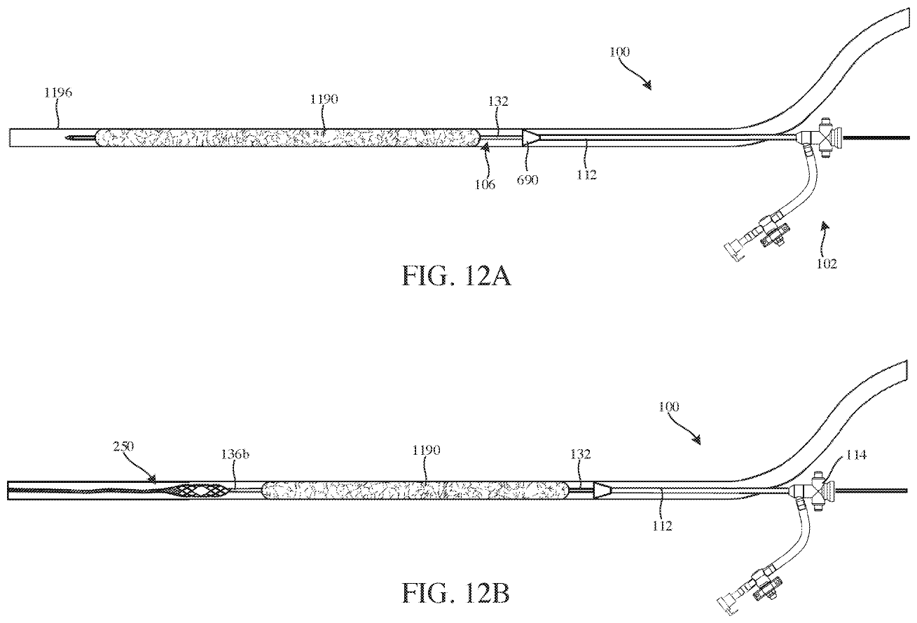

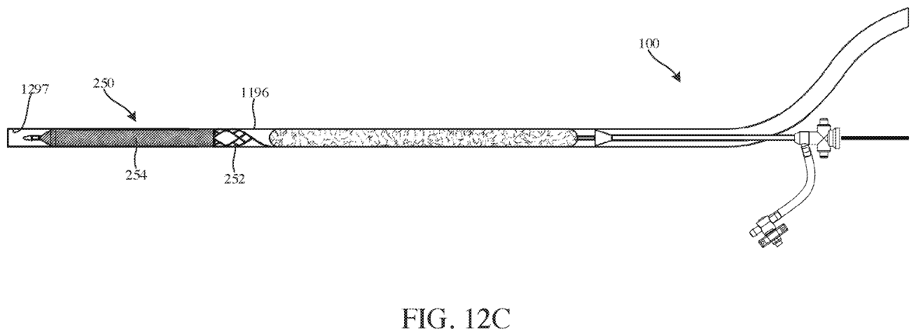

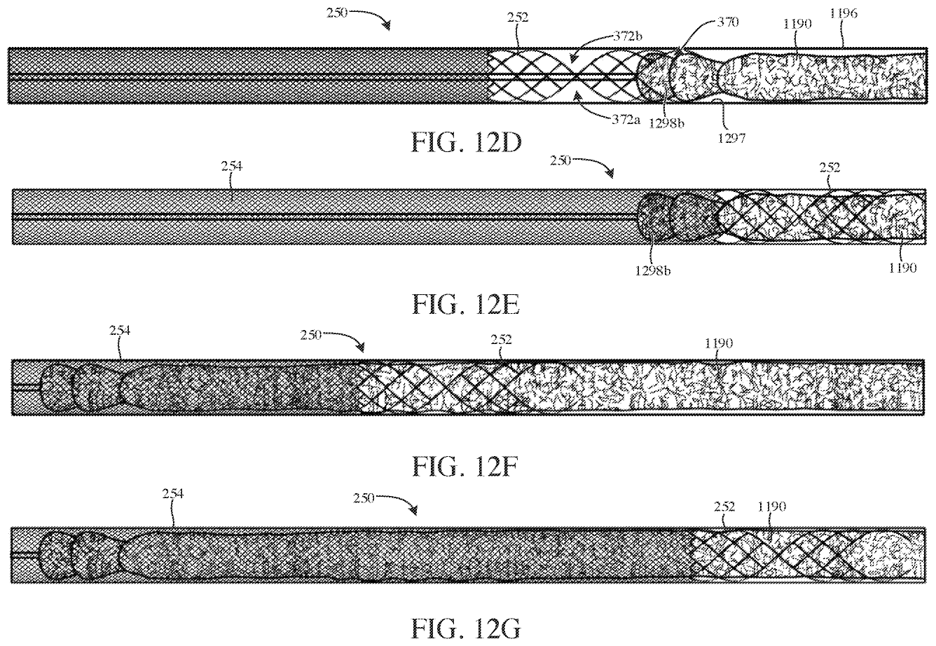

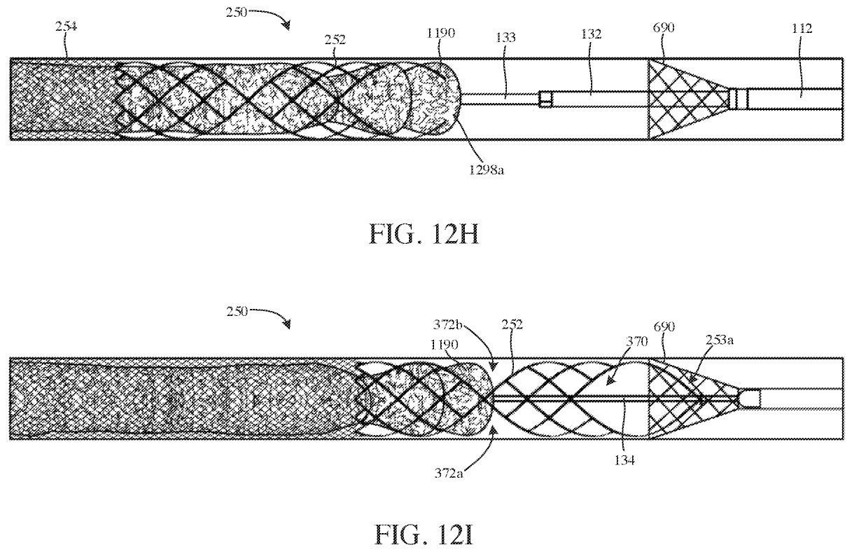

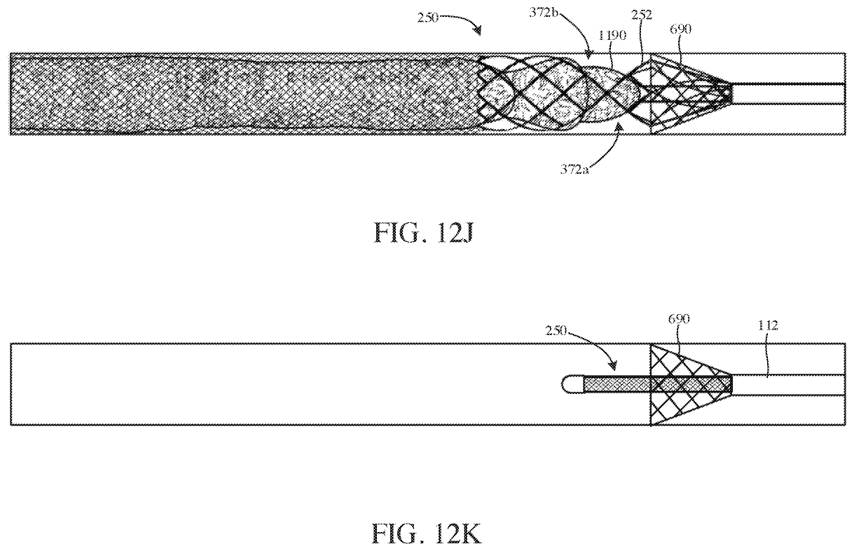

The coring element 252 is configured to core (e.g., shear, separate) thrombus from within the blood vessel when the coring element is advanced/retracted through the thrombus in the fully-expanded configuration. For example, as described in greater detail below with reference to FIGS. 12D-12K, the coring element 252 can be withdrawn proximally through the thrombus to core the thrombus. As the coring element 252 is withdrawn through the thrombus the fully-expanded diameter of the coring element 252 will flexibly adapt to match the diameter of the blood vessel. More particularly, the first and second mouths 370, 372 are configured (e.g., sized, shaped, and/or positioned) to provide most of the coring function (e.g., coring force) during operation of the coring element 252. For example, proximally-facing surfaces of the struts 367 can define a first leading edge that cuts through and cores the thrombus. Similarly, proximally-facing surfaces of the lower and upper struts 368, 369 can define a second leading edge that can also cut through and core the thrombus. In some embodiments, portions of the struts 367, the lower struts 368, and/or the upper struts 369 can be sharpened and/or can include a cutting element (e.g., a knife or knife edge) attached thereto or otherwise integrated with to further facilitate coring of the thrombus.

In one aspect of the present technology, the first mouth 370 and the second mouth 372 are longitudinally offset relative to one another. Moreover, the leading edges of the struts 367 and the leading edges of the lower and upper struts 368, 369 are oriented differently such that, for example, the first mouth 370 and the second mouth 372 are oriented at different angles when the coring element 252 is within the blood vessel. The arrangement can be more effective at coring thrombus compared to, for example, coring elements including only a single mouth (e.g., including only the first mouth 370). It is expected that the coring element 252 provides a greater coring length for engaging the wall of the blood vessel and coring (e.g., adherent) thrombus than coring elements with only a single mouth. Moreover, the coring element 252 can be relatively flexible at the first region 363 and third region 365 which include fewer struts 360 than the second region 364 and fourth region 366. For example, the coring element 252 can flex/bend at the first junctions 361 and/or the second junctions 371. In some embodiments, the first and second junctions 361, 371 enable the coring element 252 to flex in different directions (e.g., laterally and vertically). In one aspect of the present technology, this ability of the coring element 252 to flex can allow the coring element 252 to maintain a selected orientation—even when moved through tortuous vessels. In another aspect of the present technology, the arrangement of the first and second mouths 370 and 372 ensures that at least one of the first mouth 370, the first mouth portion 372a, and the second mouth portion 372b is positioned and oriented to effectively core thrombus from within the blood vessel during a thrombus extraction procedure using the coring element 252. In some embodiments, the first mouth 370 and/or the second mouth 372 can further facilitate the collapse of the coring element 252 to the non-expanded configuration.

In the embodiment illustrated in FIGS. 3A-3D, a first connection feature 374 and a second connection feature 376 are coupled to the coring element 252. As described in greater detail below with reference to FIG. 4, the intermediate shaft 133 (FIG. 1) can be operably coupled to the first connection feature 374 and the inner shaft 134 can be operably coupled to the second connection feature 376 for controlling operation (e.g., movement and expansion) of the coring element 252. In the illustrated embodiment, the first connection feature 374 is a ring coupled to the proximal portion 253a of the coring element 252 and, more specifically, to the lower first junction 361a. In other embodiments, the first connection feature 374 can be positioned on a different portion of the coring element 252 (e.g., at the upper first junction 361b, on one of the struts 367). Similarly, the second connection feature 376 can also be a ring and can be coupled to one or more of the struts 360 in the second region 364 or another region of the coring element 252. As best seen in FIG. 3D, in some embodiments the first connection feature 374 can have a diameter E₁ that is greater than a diameter E₂ of the second connection feature 376, and the first and second connection features 374, 376 can be axially aligned along an axis extending parallel to the central axis L of the coring element 252. In other embodiments, the first and second connection features 374, 376 can have other shapes and/or configurations and/or can be arranged differently relative to one another. The first and second connection features 374, 376 can be the same material as the coring element 252 or can be a different material than the coring element 252. Likewise, the first and second connection features 374, 376 can be integrally formed with the coring element 252 and/or can be attached to the coring element 252 via, for example, one or more of welds, adhesives, mechanical fasteners, and the like.

FIG. 4 is an enlarged side view of the thrombus extraction device 250 coupled to a distal portion of the thrombus extraction assembly 106 and in the fully-expanded configuration in accordance with an embodiment of the present technology. In the illustrated embodiment, the coring element 252 is coupled to the intermediate shaft 133 (e.g., to a distal portion of the intermediate shaft 133) via the first connection feature 374. In some embodiments, the coring element 252 is fixedly coupled to the intermediate shaft 133 such that movement of the intermediate shaft 133 also moves the coring element 252. The proximal portion 255a of the capture element 254 is connected to the distal portion 253b of the coring element 252. In some embodiments, the capture element 254 is formed on the distal portion 253b of the coring element 252 such that the thrombus extraction device 250 is a unitary/integral structure. For example, the capture element 254 can comprise a mesh (e.g., a braided filament mesh structure) that is woven onto the distal portion 253b of the coring element 252. In some embodiments, the distal portion 255b of the capture element 254 is coupled to the to the inner shaft 134 (e.g., to a distal portion of the inner shaft 134).

In the illustrated embodiment, the inner shaft 134 slidably extends through the second connection feature 376. That is, the inner shaft 134 can have an outer diameter that is less than the diameter E₂ (FIG. 4) of the second connection feature 376 such that the second connection feature 376 is slidable along the inner shaft 134. The inner shaft 134 can include a stop feature 478 configured to engage the second connection feature 376 of the coring element 252 to effect expansion of the coring element 252. In some embodiments, the stop feature 478 can comprise a polymeric member and/or a metallic member that is affixed to a portion of the inner shaft 134 that is distal of the second connection feature 376.

The stop feature 478 is configured (e.g., sized and shaped) to contact and engage the second connection feature 376 when the inner shaft 134 is withdrawn proximally relative to the coring element 252 via, for example, movement of the plunger 144 (FIGS. 1-3) from the first position to the second position. By this arrangement, the coring element 252 is selectively coupled to the inner shaft 134 such that the stop feature 478 can apply a proximally-directed force to the coring element 252 that can expand all or a portion of the coring element 252 to the fully-expanded configuration. For example, movement of the inner shaft 134 can forcibly expand at least the first region 363 (FIGS. 3B and 3C) of the coring element which is between the first and second connection features 374, 376. In some embodiments, the second connection feature 376 can be positioned differently with respect to the coring element 252 such that more or less of the coring element 252 is forcibly expanded when the stop feature 478 is pulled against the second connection feature 376.

In some embodiments, the capture element 254 can comprise a braided filament mesh structure, such as a braid of elastic filaments having a generally tubular, elongated portion 477 and a distal tapered portion 479. In other embodiments, the capture element 254 can be any porous structure and/or can have other suitable shapes, sizes, and configurations. Because the distal portion 255b of the capture element 254 is coupled to the inner shaft 134, axial movement of the inner shaft 134 expands/shortens and collapses/lengthens the capture element 254. For example, proximal movement of the inner shaft 134 can compress the capture element 254 along its longitudinal axis such that (i) a radius of the capture element 254 increases and (ii) the length of the capture element 254 decreases. Conversely, distal movement of the inner shaft 134 can stretch the capture element 254 along its longitudinal axis such that (i) the radius of the capture element 254 decreases and (ii) the length of the capture element 254 increases. In some embodiments, with reference to FIGS. 2A, 2B, and 4 together, distal movement of the plunger 144 can move the capture element 252 to a fully-collapsed position before the plunger 144 reaches the fully-depressed first position shown in FIG. 2A. Thus, continued distal movement of the plunger 144 (e.g., from the second position toward the first position) can pull the coring element 252 to cause the coring element 252 to collapse/longitudinally extend. That is, the plunger 144, the inner shaft 134, and the capture element 254 can collectively act to elongate/collapse the coring element 252 as the plunger 144 is distally depressed while the capture element 254 is fully collapsed. In other embodiments, the inner shaft 134 can be selectively decoupled from the capture element 254 such that proximal displacement of the inner shaft 134 expands the coring element 252 without effecting any movement of the capture element 254. In some embodiments, the capture element 254 can have a length (i) in the collapsed configuration of between about 5-30 inches (e.g., between about 10-20 inches, about 16 inches) and (ii) in the expanded configuration of between about 1-25 inches (e.g., between about 10-20 inches, about 11 inches).

In some embodiments, the capture element 254 can be formed by a braiding machine and/or a weaving machine while, in other embodiments, the capture element 254 can be manually braided and/or woven. In some embodiments, the capture element 254 is formed as a tubular braid and is then further shaped using a heat setting process. The braid can be a tubular braid of fine metal wires such as nitinol (nickel-titanium alloy), platinum, cobalt-chrome alloy, stainless steel, tungsten or titanium. In some embodiments, the capture element 254 can be formed at least in part from a cylindrical braid of elastic filaments. Thus, the braid may be radially constrained without plastic deformation such that it can self-expand on release of the radial constraint. Such a braid of elastic filaments can be referred to herein as a “self-expanding braid.” In some embodiments, the thickness of the braid filaments can be less than about 0.15 mm. In some embodiments, the braid may be fabricated from filaments and/or wires with diameters ranging from about 0.05-0.25 mm. In some embodiments, braid filaments of different diameters may be combined to impart different characteristics including: stiffness, elasticity, structure, radial force, pore size, embolic capturing or filtering ability, and so on. In some embodiments the capture element 254 and/or the coring element 252 can be coated to reduce their surface friction/abrasiveness (e.g., for arterial applications). Likewise, the capture element 254 and/or the coring element 252 can be covered with a film (e.g., via dipping or spray coating) to create a non-permeable membrane to contain clot without allowing the clot to become embedded in the interstices of the capture element 254 and/or the coring element 252, thereby facilitating ease of cleaning. In some embodiments, the number of filaments used to form the capture element 254 can be between about 20-300 (e.g., including 144 filaments, 244 filaments). In some embodiments, the size of the pores formed by the capture element 254 (e.g., in the elongated portion 477) can be between about 0.05-4.0 mm (e.g., between about 0.5 mm-2.5 mm, less than 0.4 mm).

### III. SELECTED EMBODIMENTS OF DILATOR ASSEMBLIES AND ASSOCIATED METHODS

FIGS. 5A and 5B are side views of the dilator assembly 104 of FIG. 1 in a first configuration and a second configuration, respectively, configured in accordance with embodiments of the present technology. Referring to FIGS. 5A and 5B together, the dilator assembly 104 includes a first shaft or sheath 580 extending between and operably coupling the control assembly 120 and the retention sheath 122. The dilator assembly 104 can further include a second shaft or sheath 582 slidably positioned over the first shaft 580 and operably coupled to the control assembly 120. Put differently, the second shaft 582 can define a lumen sized to slidably receive the first shaft 580 such that the first and second shafts 580, 582 are axially displaceable relative to one another. In the illustrated embodiment, the first shaft 580 is longer than the second shaft 582 such that the retention sheath 122 is positioned distal of a distal portion 583b (opposite a proximal portion 583a) of the second shaft 582. The control assembly 120 further includes a housing 595 configured to engage (e.g., mate with) the sealable hub 114 of the introducer assembly 102 (FIG. 1).

The retention sheath 122 includes a proximal portion 585a and a distal portion 585b. In the illustrated embodiment, the distal portion 585a includes an atraumatic tip 584 and the proximal portion 585a includes a first engagement feature 586. Similarly, the distal portion 583b of the second shaft 582 includes a second engagement feature 589. In some embodiments, the atraumatic tip 584 is radiopaque.

When the dilator assembly 104 is in the first configuration shown in FIG. 5A, the second shaft 582 is proximally positioned (e.g., withdrawn) relative to the first shaft 580 such that the first engagement feature 586 does not engage the second engagement feature 589. As described in greater detail below with reference to FIG. 7A, when the dilator assembly 104 is in the first configuration, the first engagement feature 586 is configured to engage (e.g., connect with, mate with) the distal portion 113b of the sheath 112 of the introducer assembly 102 (FIG. 1). In some embodiments, the engagement of the first engagement feature 586 with the sheath 112 can form a seal.

When the dilator assembly 104 is in the second configuration (FIG. 5B), the second engagement feature 589 of the second shaft 582 is configured to engage the first engagement feature 586 of the retention sheath 122. As shown, the second shaft 582 can have a diameter that is equal to or substantially equal to the outer diameter of the retention sheath 122 such that the dilator assembly 104 has a uniform or substantially uniform (e.g., smooth) outer surface in the second configuration. That is, there is no step or discontinuity in the outer surface between, for example, the first shaft 580 and the retention sheath 122. In other embodiments, the second shaft 582 and the retention sheath 122 can have different diameters, and the first and second engagement features 586, 589 can be configured to provide a smooth transition between the second shaft 582 and the retention sheath 122. In some embodiments, the engagement of the first engagement feature 586 with the second engagement feature 589 can form a seal. In some embodiments, the operator can move the dilator assembly 104 from the first configuration to the second configuration by actuating the actuator 124 of the control assembly 120 (e.g., by advancing the actuator 124 in the direction of arrows A). More specifically, as described in greater detail below with reference to FIGS. 7A-7D, actuation of the actuator 124 can distally advance (i) the first and second shafts 580, 582 together relative to the sheath 112 and then (ii) the second shaft 582 relative to the first shaft 580.

FIG. 6 is an enlarged cross-sectional side view of a portion of the thrombectomy system 100 shown in FIG. 1. More particularly, FIG. 6 shows a self-expanding funnel 690 coupled to the distal portion 113b of the sheath 112 of the introducer assembly 102 and restrained within the retention sheath 122 of the dilator assembly 104 in accordance with an embodiment of the present technology. In the illustrated embodiment, the retention sheath 122 includes a shell portion 692 coupled to the tip 584 and defining a lumen 693. In some embodiments, the shell portion 692 and the tip 584 are integrally formed together while, in other embodiments, the tip 584 can be a separate component that is coupled to the shell portion 692 by, for example, positioning at least a portion of the tip 584 in the lumen 693 and securing the shell portion 692 to the tip 584 (e.g., via an adhesive, friction fit).

The first shaft 580 of the dilator assembly 104 extends through a lumen 688 of the sheath 112 and at least partially through the lumen 693 of the shell portion 692. In the illustrated embodiment, a portion of the tip 584 snuggly receives a distal portion (e.g., a distal portion) of the first shaft 580 to secure the first shaft 580 to the retention sheath 122. In other embodiments, the first shaft 580 can be coupled to the retention sheath 122 in other manners. As further shown in FIG. 6, the first shaft 580 and the tip 584 can define a continuous lumen 691 for receiving a guidewire (not shown). In some embodiments, the guidewire can have a diameter of about 0.038 inch, 0.035 inch, about 0.018 inch, 0.014 inch, greater than about 0.38 inch, less than about 0.1 inch, or less than about 0.05 inch.

In the illustrated embodiment, an inner diameter F₁ of the shell portion 692 is greater than an external diameter F₂ of the first shaft 580 such that an annular retaining/receiving space 694 is formed between the outer surface of the first shaft 580 and the inner surface of the shell portion 692. The receiving space 694 is configured (e.g., sized and shaped) to receive and/or retain the funnel 690 in a constrained configuration. Accordingly, in some embodiments the funnel 690 can have a diameter substantially matching the inner diameter F₁ of the shell portion 692 when the funnel 690 is in the constrained configuration. In some embodiments, the first engagement feature 586 of the retention sheath 122 can engage (e.g., sealingly engage) the distal portion 113b of the sheath 112 when the funnel 690 is retained within the retention sheath 122.

FIGS. 7A-7D are side views illustrating various stages in a process or method for deploying the funnel 690 in accordance with embodiments of the present technology. Referring first to FIG. 7A, the dilator assembly 104 is initially positioned within the introducer assembly 102 in the first configuration (FIG. 5A) such that (i) the housing 595 of the control assembly 120 is coupled to/engages the sealable hub 114 and (ii) the first engagement feature 586 of the retention sheath 122 sealingly engages the distal portion 113b of the sheath 112. In other embodiments, the first engagement feature 586 need not sealingly engage the sheath 112. In the initial position shown in FIG. 7A, the actuator 124 of the control assembly 120 is in a first position (e.g., a fully-retracted position) and the funnel 690 is contained in the constrained configuration within the retention sheath 122, as shown in FIG. 6.

In the arrangement shown in FIG. 7A, the introducer assembly 102 and the dilator assembly 104 (collectively “assemblies 102, 104”) can be used to percutaneously access a venous vessel of a patient through, for example an access site such as a popliteal access site, a femoral access site, an internal jugular access site, and/or other access site. In some embodiments, the assemblies 102, 104 are inserted through another introducer sheath (not shown). In some embodiments, the assemblies 102, 104 are advanced within the venous vessel to a treatment position in which the distal portion 113b of the sheath 112 is proximate to (e.g., proximal of) a thrombus in the venous vessel.

Referring to FIG. 7B, after positioning the assemblies 102, 104, the funnel 690 (shown as transparent in FIGS. 7B and 7C for the sake of clarity) can be deployed by, for example, moving the actuator 124 from the first position (FIG. 7B) to a second position (e.g., an intermediate position, a mid-stroke position, a drop-off position) to distally advance the first and second shafts 580, 582 together relative to the sheath 112. The distal advancement of the first shaft 580 causes the retention sheath 122 to move distally over and away from the funnel 690. The funnel 690 self-expands to an expanded (e.g., unconstrained) configuration when the funnel 690 is no longer constrained by the retention sheath 122. In other embodiments, the control assembly 120 is configured such that moving the actuator 124 from the first position to the second position distally advances only the first shaft 580 of the dilator assembly 104 rather than both the first and second shafts 580, 582 together.

The funnel 690 can comprise a variety of shapes and sizes and can be made from a variety of materials. In some embodiments, in the expanded configuration, the funnel 690 can have (i) a maximum diameter greater than and/or equal to the diameter D of the coring element 252 (FIGS. 3B and 3C) when the coring element 252 is in the fully-expanded configuration and (ii) a minimum diameter substantially equal to an outer diameter of the sheath 112. In some embodiments, the funnel 690 can have a length N that is greater than and/or equal to the length M of the coring element 252 (FIGS. 3A-3D) such that the coring element 252 can be received and contained within the funnel 690. In other embodiments, the length N of the funnel 690 can be less than the length M of the coring element 252. In some embodiments, the funnel 690 can have a conically shaped portion, and specifically, a truncated-conically shaped portion. In some embodiments, the funnel 690 can be formed from at least one of a castellated nitinol braid, a nitinol braided stent, a laser cut nitinol, a laser cut polymer tube, an injection molded polymeric structure, or an inflatable balloon. In some embodiments, the funnel 690 can comprise a mesh having a pore size sufficiently small to prevent the passage of thrombus through the pores of the mesh. In some embodiments, the funnel 690 can be permeable to blood.

Referring to FIG. 7C, after the funnel 690 has been deployed, the dilator assembly 104 can be moved to the second configuration (FIG. 5B). For example, the operator can move the actuator 124 of the control assembly 120 from the second position (FIG. 7B) to a third position (e.g., a fully-advanced position) to distally advance the second shaft 582 relative to the first shaft 580 until the second engagement feature 589 of the second shaft 582 engages the first engagement feature 586 of the retention sheath 122. As shown in FIG. 7D, after moving the dilator assembly 104 to the second configuration, the dilator assembly 104 can be fully retracted and withdrawn from the introducer assembly 102. For example, the dilator assembly 104 can be proximally retracted through the lumen of the sheath 112 and out of the sealable hub 114 of the introducer assembly 102.

Referring to FIGS. 7A-7D together, in one aspect of the present technology, moving the dilator assembly 104 to the second configuration before retracting the dilator assembly 104 from the introducer assembly 102 can inhibit or even prevent the dilator assembly 104 from damaging the funnel 690 or other components of the introducer assembly 102 during retraction of the dilator assembly 104. More specifically, if the dilator assembly 104 did not include the second shaft 582, proximal retraction of the retention sheath 122 into the sheath 112 could cause the retention sheath 122 (e.g., the first engagement feature 586) to snag or damage the deployed funnel 690. However, because the second shaft 582 has a diameter that is equal to or substantially equal to the outer diameter of the retention sheath 122, the dilator assembly 104 has a uniform or substantially uniform (e.g., smooth) outer surface in the second configuration, and is therefore less likely to snag or otherwise damage the funnel 690, the sealable hub 114, and/or other components of the introducer assembly 102 during retraction. In other embodiments, the second shaft 582 and the retention sheath 122 can have different diameters, and the first and second engagement features 586, 589 can be configured to provide a smooth transition between the second shaft 582 and the retention sheath 122.

In another aspect of the present technology, the movement of the actuator 124 from the first position to the third position both (i) advances the first and second shafts 580, 582 together to deploy the funnel 690 (e.g., as the actuator 124 moves from the first position to the second position) and (ii) advances the second shaft 582 relative to the first shaft 580 (e.g., as the actuator 124 moves from the second position to the third position) so that the dilator assembly 104 has a generally uniform outer diameter. This “dual-action” allows the control assembly 120 to be coupled to the sealable hub 114 during both the deployment of the funnel 690 and the advancement of the second shaft 582 toward the first shaft 580. This can advantageously inhibit or prevent the inadvertent advancement of the retention sheath 122 and therefore the premature deployment of the funnel 690. For example, the dilator assembly 104 and the introducer assembly 102 must often be fully removed from the patient for reloading of the funnel 690 if the funnel 690 is prematurely deployed—potentially increasing the trauma to the patient and the duration of the thrombectomy procedure. In contrast, some conventional dilator assemblies include a dilator that is “floating” (e.g., not locked to or engaged with an introducer assembly) such that an inadvertent bump or other force on the dilator assembly can cause corresponding movement of the dilator assembly.

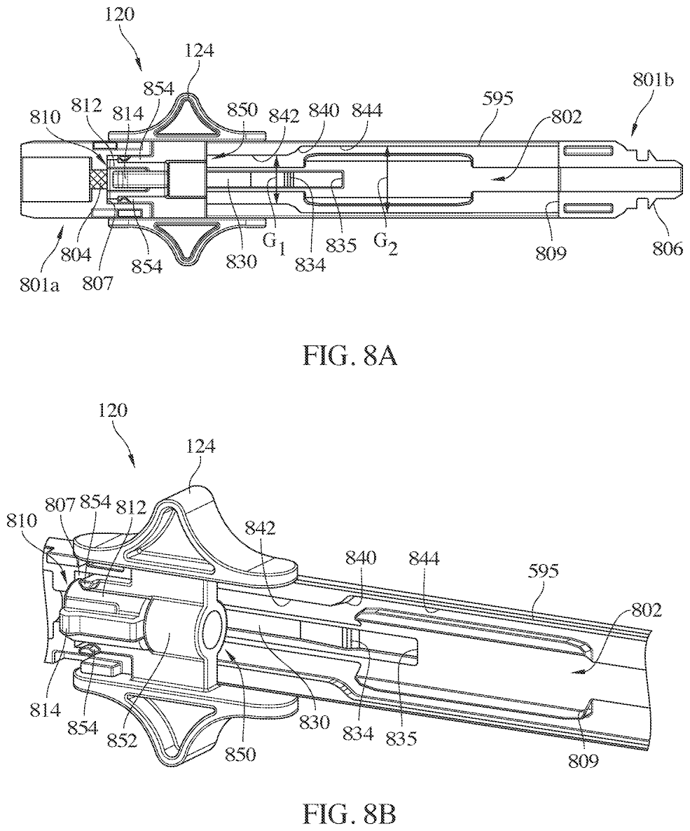

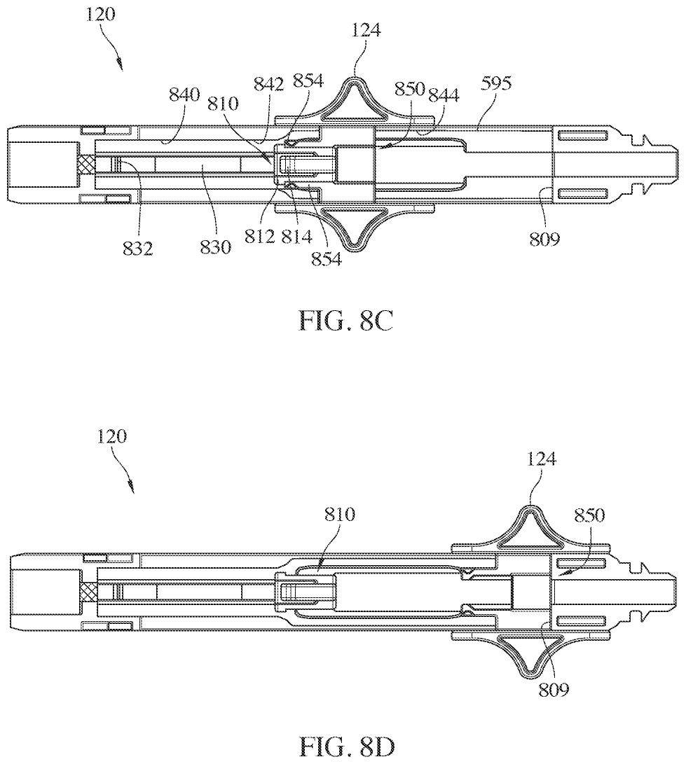

FIGS. 8A, 8C, and 8D are cross-sectional side views, and FIG. 8B is an enlarged cross-sectional isometric view, of the control assembly 120 configured in accordance with embodiments of the present technology. In FIGS. 8A and 8B the actuator 124 is in the first position shown in FIG. 7A, in FIG. 8C the actuator 124 is in the second position shown in FIG. 7B, and in FIG. 8D the actuator 124 is in the third position shown in FIG. 7C.

Referring first to FIG. 8A, the control assembly 120 includes a proximal portion 801a and a distal portion 801b and defines a lumen 802 extending therethrough between the proximal and distal portions 801a, b. In the illustrated embodiment, the control assembly 120 includes a sealable member 804 at or proximate the proximal portion 801a and a connection portion 806 at or proximate the distal portion 801b. The sealable member 804 can be configured to selectively seal the lumen 802 of the control assembly 120 and, in some embodiments, can receive a guidewire (not shown) therethrough. The connection portion 806 is configured to mate/engage with the sealable hub 114 of the introducer assembly 102 to secure the control assembly 120 thereto, as described in detail above with reference to FIGS. 7A-7C. For example, in some embodiments the connection portion 806 can include a snap feature (e.g., having one or more teeth, flanges), a twist lock (e.g., a bayonet- or luer-type fitting), and/or other feature for engaging and/or locking to the sealable hub 114.

Referring to FIGS. 8A and 8B together, in the illustrated embodiment the control assembly 120 further includes a first shaft hub 810 and a second shaft hub 850. The first shaft hub 810 is configured to be coupled to the first shaft 580 of the dilator assembly 104, and the second shaft hub 850 is configured to be coupled to the second shaft 582 of the dilator assembly 104. The first and second shafts 580, 582 are not shown in FIGS. 8A-8D for the sake of clarity. In the illustrated embodiment, the second shaft hub 850 is connected to (e.g., integrally formed with) the actuator 124, which extends outside the housing 595 and is configured to be advanced distally and/or retracted proximally by the operator. The first shaft hub 810 includes a first body portion 812 and one or more first engagement or snap features 814 (only one first engagement feature 814 is visible in FIGS. 8A-8D) that extend radially and/or axially away from the first body portion 812 and into a corresponding first track 830 formed in the housing 595. The second shaft hub 850 similarly includes a second body portion 852 and second engagement or snap features 854 (e.g., a pair of similar or identical second engagement features 854) that extend radially and/or axially away from the second body portion 852 and into a corresponding second track 840 formed in the housing 595.

In the illustrated embodiment, the first track 830 includes one or more proximal detents 832 (obscured in FIGS. 8A and 8B; shown in FIG. 8C), one or more distal detents 834, and a distal terminus 835. In some embodiments, the first track 830 can include a pair of opposing (e.g., radially opposite) proximal detents 832 and a pair of opposing distal detents 834. The second track 840 includes a first portion 842 having a first track width or height G₁ (FIG. 8A) and a second portion 844 having a second track width or height G₂ (FIG. 8A) greater than the first track width G₁. In some embodiments, the transition (e.g., a slope or step) between the first and second portions 842, 844 of the second track 840 is generally aligned over and/or proximate to the distal detents 834 of the first tack 830.

In operation, the first and second shaft hubs 810, 850 are configured to slide within the lumen 802 along the first and second tracks 830, 840, respectively. In some embodiments, the first engagement features 814 and/or the second engagement features 854 are flexible such that they can flex/bend as the first and second shaft hubs 810, 850 move along the first and second tracks 830, 840. The configuration/arrangement of the first and second shaft hubs 810, 850 and the first and second tracks 830, 840—for example, the arrangement of the proximal and distal detents 832, 834, the first portion 842, and/or the second portion 844—can facilitate the movement of the dilator assembly 104 from the first configuration (FIG. 5A) to the second configuration (FIG. 5B).

More specifically, in the first position shown in FIGS. 8A and 8B, the actuator 124 is positioned at a most proximal position along the housing 595. For example, the first shaft hub 810 can abut a proximal wall portion 807 of the housing 595. In the first position, the first portion 842 of the second track 840 compresses (e.g., presses, constrains) the second engagement features 854 of the second shaft hub 850 radially inward toward and into engagement with the first shaft hub 810 (e.g., with the first body portion 812). Put differently, a distance (e.g., diameter) of the second shaft hub 850 between the second engagement features 854 can be greater than the first diameter G₁ when the second shaft hub 850 is in a relaxed state, unconstrained by the first portion 842 of the second track 840. By this arrangement, the second shaft hub 850 is secured to the first shaft hub 810 such that movement of the actuator 124 along the first portion 842 of the second track 840 moves both the first and second shaft hubs 810, 850. In some embodiments, the first body portion 812 of the first shaft hub 810 can include various features (e.g., grooves, channels, teeth) for mating with the second engagement features 854 of the second shaft hub 850 to thereby secure the first and second shaft hubs 810, 850 together.

Moreover, in the first position, at least a portion of the first engagement features 814 of the first shaft hub 810 can be positioned proximal of the proximal detents 832 (FIGS. 8C and 8D). The proximal detents 832 can thus retain the first shaft hub 810—and the second shaft hub 850 and the actuator 124 secured thereto—in the first position until a predetermined force is applied to the actuator 124 in the distal direction. In one aspect of the present technology, this arrangement can inhibit the unintended distal advancement of the first shaft 580—and thus the premature deployment of the funnel 690 (FIGS. 7A-7C). In some embodiments, when the predetermined force is applied to the actuator 124, the first engagement features 814 flex inwardly such that first shaft hub 810 can slide distally thereby.

Accordingly, referring to FIGS. 8A-8C together, the actuator 124 can be advanced distally from the first position to the second position shown in 8C after the first engagement features 814 disengage the proximal detents 832. As the actuator 124 is moved distally, the first and second shaft hubs 810, 850 move distally together—thereby advancing the first and second shafts 580, 582 together as shown in FIG. 7B—until the first shaft hub 810 reaches the distal terminus 835 of the first track 830 and/or the second shaft hub 840 reaches the second portion 844 of the second track 840. More specifically, the distal terminus 835 and/or the distal detents 834 of the first track 830 can engage the first engagement features 814 to prevent the first shaft hub 810 (and thus the first shaft 580) from moving farther distally. At the same time, the greater-diameter second portion 844 of the second track 840 allows the second engagement features 854 to move radially outward (e.g., flex radially outward toward the relaxed state) and out of engagement with first shaft hub 810. That is, the control assembly 120 is configured such that the second engagement features 854 of the second shaft hub 850 reach the transition point between the first and second portions 842, 844 of the first track 840 at substantially the same time as the first engagement features 814 of the first shaft hub 810 reach/engage the distal detents 834 of the first track 830.

Accordingly, as shown in FIG. 8D, the second shaft hub 850 can leave the first shaft hub 810 behind and advance further distally to the third position. As the second shaft hub 850 moves distally while the first shaft hub 810 remains stationary, the second shaft 582 is advanced distally toward the retention sheath 122 as shown in FIG. 7C. In some embodiments, the second shaft hub 850 can abut a distal wall portion 809 of the housing 595 in the third position.

Referring to FIGS. 5A-8D together, in one aspect of the present technology, the control assembly 120 facilitates the movement of the dilator assembly 104 from the first configuration to the second configuration with only as a single movement of the actuator 124 from the first to third positions. As described above, this advantageously allows the control assembly 120 to be coupled to the sealable hub 114 at all times during deployment of the funnel 690, which controls deployment of the funnel 690 and prevents the funnel 690 from inadvertently being deployed. This is expected to reduce the potential for the other components of the system, such as the retention sheath 122, from catching on the funnel 690 as the dilator is retracted through the sheath 112. Moreover, deployment of the funnel 690 and advancement of the second shaft 582 are achieved by a single stroke and are thus greatly simplified.

In some embodiments, the actuator 124 can be moved proximally (e.g., from the third position toward the first position) to facilitate loading of the funnel 690. For example, the dilator assembly 104 can be inserted into the sheath 112 when the control assembly 120 is in the third position such that the retention sheath 122 extends from the distal portion 113b of the sheath 112 and distally beyond the funnel 690. The operator can then move the actuator 124 to the second position, thereby forcing the second shaft hub 850 into engagement with the first shaft hub 810 via the narrowing of the second track 840 from the second portion 844 to the first portion 842. The loading tool 108 (FIG. 1) can then be slid proximally over the retention sheath 122 and the funnel 690 until the funnel 690 is fully encapsulated by the loading tool 108 and/or until the funnel 690 is in the constrained configuration. The operator can then move the actuator 124 from the second position to the first position to retract the retention sheath 122 over the funnel 690 to thereby load/capture the funnel 690 within the receiving space 694 of the retention sheath 122. Finally, the loading tool 108 can be removed.

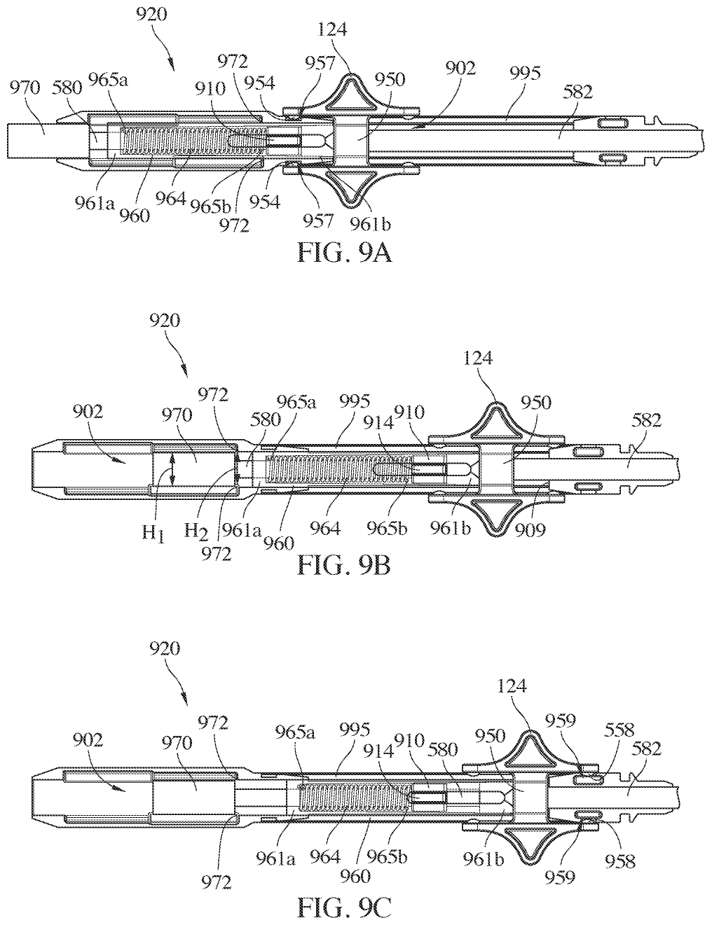

In other embodiments, control assemblies in accordance with the present technology can include other components and/or configurations for facilitating the dual-action of (i) advancing the first and second shafts 580, 582 to deploy the funnel 690 and (ii) advancing the second shaft 582 relative to the first shaft 580 to provide a uniform outer surface that facilitates retraction of the dilator assembly 104. FIGS. 9A-9C, for example, are cross-sectional side views of a control assembly 920 including the actuator 124 in the first position, the second position, and the third position (FIGS. 7A-7C) configured in accordance with another embodiment of the present technology.

The control assembly 920 can include some features generally similar to the control assembly 120 described in detail above with reference to FIGS. 8A-8D. For example, referring to FIGS. 9A-9C together, the control assembly 920 includes a first shaft hub 910 coupled to the first shaft 580 of the dilator assembly 104, and a second shaft hub 950 coupled to the second shaft 582 of the dilator assembly 104. In the illustrated embodiment, the second shaft hub 950 is connected to (e.g., integrally formed with) the actuator 124, which extends outside a housing 995 of the control assembly 920 and is configured to be advanced distally and/or retracted proximally by the operator. The first and second shaft hubs 910, 950 are configured to slide at least partially through a lumen 902 extending through the housing 995.

In the illustrated embodiment, the control assembly 920 further includes an elongate member 960 (shown as transparent in FIGS. 9A-9C for the sake of clarity) having (i) a proximal portion 961a positioned proximal of the first shaft hub 910 and (ii) a distal portion 961b positioned distal of the first shaft hub 910 and coupled to the second shaft hub 950. The first shaft hub 910 can be slidably positioned within the elongate member 960. A biasing member 964, such as a compression spring, extends between the proximal portion 961a of the elongate member 960 and the first shaft hub 910. In some embodiments, a proximal portion 965a of the biasing member 964 is connected to the proximal portion 961a of the elongate member 960 and a distal portion 965b of the biasing member 964 is connected to the first shaft hub 910.

The control assembly 920 can further include a stop member 970 coupled to the first shaft 580 (e.g., to a proximal portion of the first shaft 580). The stop member 970 is configured to slide at least partially through the lumen 902 of the housing during operation of the control assembly 920 and can be fully contained within the housing 995 (e.g., as shown in FIGS. 9B and 9C) and/or can extend fully or partially outside of the housing 995 (e.g., as shown in FIG. 9A). As shown in FIG. 9B, the stop member 970 has a dimension (e.g., diameter) H₁ that is greater than a dimension H₂ of a stop portion 972 of the housing 995. By this arrangement, the stop member 970 is configured to contact the stop portion 972 of the housing 995 to thereby prevent the first shaft 580 (and the retention sheath 122 attached thereto) from advancing farther distally.

Referring to FIG. 9A, in the first position, the first shaft hub 910 engages (e.g., mates with) the second shaft hub 950 such that distal advancement of the actuator 124 moves both the first and second shaft hubs 910, 950. Moreover, the biasing member 964 is at equilibrium and thus does not exert any force on, for example, the first shaft hub 910. In some embodiments, the actuator 124 and/or the second shaft hub 950 can include first engagement features 954 (e.g., bumps, projections) that can engage (e.g., mate with) corresponding first detents 957 in the housing 995 to releasably secure the actuator 124 in the first position until a predetermined force is applied to the actuator in the distal direction. In some embodiments, when the predetermined force is applied to the actuator 124, the first engagement features 954 can flex outwardly and out of the first detents 957 to permit the first and second shaft hubs 910, 950 to move distally.

Accordingly, referring to FIGS. 9A and 9B together, the actuator 124 can be advanced distally from the first position to the second position after the first engagement features 954 disengage the first detents 957. As the actuator 124 is moved distally, the first and second shaft hubs 910, 950 move distally together—thereby advancing the first and second shafts 580, 582 together as shown in FIG. 7B—until the stop member 970 reaches and contacts the stop portion 972 of the housing 995. More specifically, the biasing member 964 can exert a force against the first shaft hub 910 to move the first shaft hub 910 together with the second shaft hub 950. When the stop member 970 contacts the stop portion 972, the first shaft hub 910 is stopped from advancing farther distally.

Accordingly, referring to FIGS. 9B and 9C together, the second shaft hub 950 can leave the first shaft hub 910 behind as the actuator 124 is moved farther distally to the third position. As the second shaft hub 950 moves distally while the first shaft hub 910 remains stationary, the second shaft 582 is advanced distally toward the retention sheath 122 as shown in FIG. 7C. In some embodiments, the second shaft hub 950 can abut a distal wall portion 909 of the housing 995 in the third position, which prevents the second shaft hub 950 from advancing farther. As further shown in FIG. 9C, advancing the second shaft hub 950 to the third position compresses the biasing member 964 between the first shaft hub 910, which remains stationary, and the proximal portion 961a of the elongate member 960 which continues to move with the second shaft hub 950. In some embodiments, the bias force exerted by the biasing member 964 can facilitate the subsequent movement of the actuator 124 from the third position to the second position. In some embodiments, the actuator 124 can include second engagement features 958 (e.g., bumps, projections) that can engage (e.g., mate with) corresponding second detents 959 in the housing 995 to releasably secure the actuator 124 in the third position until a predetermined force is applied to the actuator in the proximal direction. In some embodiments, this force can be less than that required to disengage the first engagement features 954 from the first detents 957 due to the biasing force of the biasing member 964. In other embodiments, the detents 959 can comprise a track (e.g., an L-shaped track), and the second shaft hub 950 can be rotated to rotate the second engagement features 958 into the track to releasably secure the actuator 124 in the third position.

In other embodiments, the stop member 970 is not configured to stop distal advancement of the first shaft 580. Rather, the stop member 970 can instead be a luer flush port 970 (or another component) that simply moves together with the first shaft 580, or can be omitted altogether. In such embodiments, the first shaft hub 910 can move along a track (not shown) formed in the housing 995 in a similar manner as the first shaft hub 810 described in detail with reference to FIGS. 8A-8D. For example, the first shaft hub 910 can include first engagement or snap features 914 (only one first engagement feature 914 is visible in FIGS. 9A-9C) that extend (i) radially and/or axially away from a body portion of the first shaft hub 910, (ii) out of the elongate member 960, and (iii) into the track in the housing 995. The track can include a detent or other feature (not shown) configured (e.g., positioned and shaped) to stop the first shaft hub 910 from moving farther distally when the first shaft hub 910 reaches the second position shown in FIG. 9B.

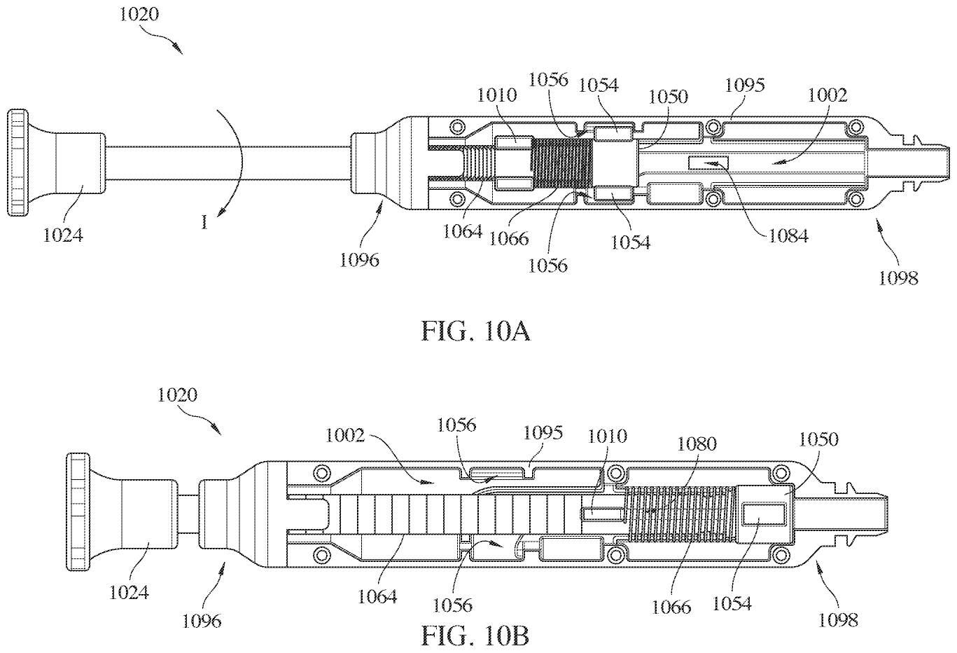

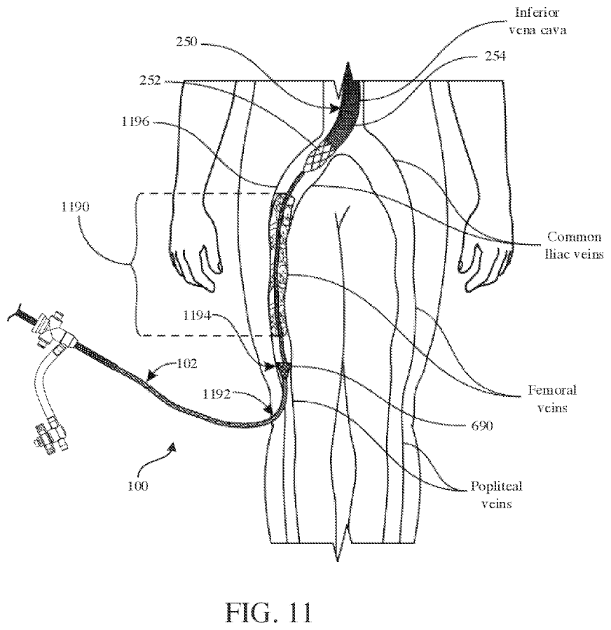

FIGS. 10A and 10B are partially cross-sectional side views of a control assembly 1020 configured in accordance with another embodiment of the present technology. In general, the control assembly is movable between (i) the first position (shown in FIG. 10A) in which the second shaft 582 is retracted proximally relative to the first shaft 580 as shown in FIGS. 5A and 7A and (ii) the third position (shown in FIG. 10B) in which the second shaft 582 is advanced distally relative to the first shaft 580 to form a generally uniform outer surface of the dilator assembly 104 as shown in FIGS. 5B and 7C. In one aspect of the present technology, the control assembly 1020 does not include the intermediate second position (FIG. 7B), but instead fluidly moves between the first and third positions.

The control assembly 1020 can include some features generally similar to the control assembly 120 and/or the control assembly 920 described in detail above with reference to FIGS. 8A-9C. For example, referring to FIGS. 10A and 10B together, the control assembly 1020 includes an actuator 1024 (e.g., a plunger 1024) that is movable relative to/through a lumen 1002 of a housing 1095. The plunger 1024 is coupled to (i) a first shaft hub 1010 that is coupled to the first shaft 580 of the dilator assembly 104 and (ii) a second shaft hub 1050 that is coupled to the second shaft 582 of the dilator assembly 104. The first and second shafts 580, 582 are not shown in FIGS. 10A and 10B for the sake of clarity.

In the illustrated embodiment, the second shaft hub 1050 includes engagement features 1054 that are configured (e.g., sized and shaped) to engage with a corresponding stop portion 1056 formed in the housing 1095 when the plunger 1024 is in the first position shown in FIG. 10A. The first shaft hub 1010 is configured to slide along a track 1080 formed in/along a portion of the plunger 1024. In some embodiments, the track 1080 includes at least one detent 1084 at a distal portion thereof and configured to stop/block distal advancement of the first shaft hub 1010. In other embodiments, the housing 1095 can include a flange or other component configured to stop distal advancement of the first shaft hub 1010.

A first biasing member 1064 (e.g., a compression spring) extends between and operably couples (e.g., connects) the first shaft hub 1010 and a proximal portion 1096 of the housing 1095. A second biasing member 1066 (e.g., a compression spring) extends between and operably couples (e.g., connects) the first and second shaft hubs 1010, 1050. In the first position shown in FIG. 10A, both of the first and second biasing members 1064, 1066 are compressed and under load and therefore urge the first and second shaft hubs 1010, 1050, respectively, distally. In some embodiments, the first biasing member 1064 has a larger compression force than the second biasing member 1066.

In the first position shown in FIG. 10A, the plunger 1024 is locked in a proximally retracted position by the engagement of the engagement features 1054 with the stop portion 1056 of the housing 1095. To move the control assembly 1020 to the third position shown in FIG. 10B, the operator can rotate the plunger 1024 (e.g., as indicated by arrow I in FIG. 10A) to unlock the second shaft hub 1050 and the plunger 1024. When the plunger 1024 is unlocked, the first biasing member 1064 is configured to drive the first shaft hub 1010 distally until the first shaft hub 1010 is stopped by/within the detent 1084. In one aspect of the present technology, because the first biasing member 1064 is stronger than the second biasing member 1066, the second biasing member 1066 remains substantially compressed until the first shaft hub 1010 engages the detent 1084. Therefore, both the first and second shaft hubs 1010, 1050—and thus both the first and second shafts 580, 582—move together until the first shaft hub 1010 reaches the detent 1084. Then, the second biasing member 1066 is configured to drive the second shaft hub 1050 distally relative to the first shaft hub 1010 (e.g., away from the first shaft hub 1010). In the third position shown in FIG. 10B, the first and second biasing members 1064, 1066 can bias the first and second shaft hubs 1010, 1050 distally to maintain the control assembly 1020 in the third position. By this arrangement, the first and second shafts 580, 582 are automatically moved from the first configuration (FIG. 5A) to the second configuration (FIG. 5B)—deploying the funnel and readying the dilator assembly 104 for retraction as shown in FIGS. 7A-7D.

In other embodiments, the first and second biasing members 1064, 1066 can be arranged in an opposite configuration. For example, the first biasing member 1064 can extend between and operably couple the first and second shaft hubs 1010, 1050, and the second biasing member 1066 can extend between and operably couple the second shaft hub 1050 and a distal portion 1098 of the housing 1096. Likewise, the second biasing member 1066 can have a larger compression force than the first biasing member 1064. Thus, the first and second biasing members 1064, 1066 can bias the control assembly 1020 to the first position. To move the control assembly 1020 to the third position, the user can advance the plunger 1024 against the compression forces of the first and second biasing members 1064, 1066 until the second shaft hub 1050 reaches the third position. In some embodiments, the user can then rotate the plunger 1024 to lock the control assembly 1020 in the third position.

### IV. SELECTED EMBODIMENTS OF THROMBECTOMY METHODS

FIG. 11 is a schematic view of an introduction technique for accessing a thrombus 1190 for treatment with the thrombectomy system 100 in accordance with an embodiment of the present technology. The thrombus 1190 (e.g., clot material) can be located in a blood vessel 1196 and accessed through an access site 1192 such as the popliteal access site, or other venous or arterial access sites. The introducer assembly 102 can extend from the popliteal access site 1192, or other venous or arterial access sites, to a deployment position 1194 at which the self-expanding funnel 690 can be deployed and which can be proximate to the thrombus 1190. As described in greater detail below with reference to FIGS. 12A-12K, the thrombus extraction device 250 can be passed through the thrombus 1190 in the direction of blood flow and then retracted through the thrombus 1190 in a direction with blood flow. During retraction, the coring element 252 can core/separate the thrombus 1190 and the capture element 254 can capture all or a portion of the thrombus 1190. In some embodiments, some or all of the thrombus extraction device 250 can extend into one of the iliac veins and/or the inferior vena cava.

More particularly, FIGS. 12A-12C are side views, and FIGS. 12D-12M are enlarged side views, of the thrombectomy system 100 positioned within the blood vessel 1196 during a thrombectomy procedure to treat (e.g., remove) the thrombus 1190 in accordance with embodiments of the present technology.

FIG. 12A illustrates the thrombectomy system 100 intravascularly positioned within the blood vessel 1196 after (i) deploying the self-expanding funnel 690 (e.g., as described in detail with reference to FIGS. 5A-10B), (ii) removing the dilator assembly 104 from the introducer assembly 102, and (iii) advancing the outer shaft 132 of the thrombus extraction assembly 106 through the sheath 112 and the thrombus 1190. The distal advance of the outer shaft 132 through the thrombus 1190 can be either with or against the direction of blood flow.

FIG. 12B illustrates the thrombectomy system 100 after advancing the thrombus extraction device 250 through the outer shaft 132 to a deployed position distal of the thrombus 1190. In some embodiments, the thrombus extraction device 250 can be constrained within the outer shaft 132 and inserted, together with the outer shaft 132, into the lumen of the sheath 112 via the sealable hub 114. In some embodiments, the thrombus extraction device 250 can be deployed by advancing the thrombus extraction device 250 beyond the distal portion 136b of the sheath 112 and/or by retracting the outer shaft 132 relative to the thrombus extraction device 250 until the thrombus extraction device 250 is beyond the distal portion 136b of the outer shaft 132.

FIG. 12C illustrates the thrombectomy system 100 after fully-expanding the thrombus extraction device 250. In some embodiments, at least a portion of the coring element 252 and/or the capture element 254 contact a wall 1297 of the blood vessel 1196 in the fully-expanded position. As described in detail above with reference to FIGS. 2A and 2B, in some embodiments the thrombus extraction device 250 can be fully expanded by moving the plunger 144 from the first position to the second position and securing the plunger 144 in the second position to thereby fix the relative position of the inner shaft 134 with respect to the intermediate shaft 133.

In general, FIGS. 12D-12K illustrate the proximal retraction of the thrombus extraction device 250 through the thrombus 1190 to capture at least a portion of the thrombus 1190, and the subsequent joint retraction of the thrombus extraction device 250 and the captured thrombus 1190 into the funnel 690 and the sheath 112.

Referring first to FIG. 12D, proximal retraction of the thrombus extraction device 250 causes the coring element 252 to separate and/or core a distal portion 1298b of the thrombus 1190 from the wall 1297 of the blood vessel 1196. As shown in FIG. 12E, continued proximal retraction of the thrombus extraction device 250 through the thrombus 1190 causes the capture element 254 to capture the distal portion 1298b of the thrombus 1190 therein. FIGS. 12F-12H illustrate further proximal retraction of the thrombus extraction device 250 which causes further separation, coring, and/or capture of the thrombus 1190. As seen in FIG. 12H, a proximal portion 1298a of the thrombus 1190 is cored and captured as the thrombus extraction device 250 is proximally retracted toward the funnel 690 and the sheath 112.

As described in detail above with reference to FIGS. 3A-4, the coring element 252 can include both the first mouth 370 and the second mouth 372 (identified in FIG. 12D). Thus, the first mouth 370, the first mouth portion 372a, and/or the second mouth portion 372b can facilitate the coring/separating of the thrombus 1190 during proximal retraction of the thrombus extraction device 250. In one aspect of the present technology, the first mouth 370 and the second mouth 372 are radially offset relative to one another which can increase the coring effectiveness—even when the blood vessel 1196 is very tortuous and/or the thrombus 1190 is strongly adhered to the wall 1297 of the blood vessel 1196—by ensuring that at least one of the first mouth 370 and the second mouth 372 is positioned and oriented to effectively core the thrombus 1190.

In some embodiments, as shown in FIGS. 121 and 12G, the thrombus extraction device 250 can be proximally retracted until the proximal portion 253a of the coring element 252 is contained (e.g., positioned) within the funnel 690. More specifically, the thrombus extraction device 250 can be proximally retracted until all or a portion of the first mouth 370 and/or the second mouth 372 of the coring element 252 are contained within the funnel 690. In some embodiments, when one or both of the first and second mouths 370, 372 are positioned within the funnel 690, the thrombus extraction device 250 can be moved or transformed from the expanded deployed state to the compressed state to compress and secure the thrombus 1190 captured by the thrombus extraction device 250. In some embodiments, for example, the intermediate shaft 133 (FIG. 12H) can be unlocked and/or decoupled from the inner shaft 134 (e.g., via user actuation of the plunger 144 shown in FIGS. 1-2B) such that the inner shaft 134 can be advanced distally relative to the intermediate shaft 133 to collapse or compress the thrombus extraction device 250.

After the thrombus extraction device 250 has been collapsed, the thrombus extraction device 250 can be proximally retracted through the funnel 690 and into the sheath 112 as depicted in FIG. 12K. The thrombus extraction device 250 can continue to be proximally retracted until the thrombus extraction device 250 and the captured thrombus 1190 are fully contained within the sheath 112. In some embodiments, the thrombus extraction device 250 and the captured thrombus 1190 can then be withdrawn through the sheath 112 and the sealable hub 114 (FIG. 12B).

In some embodiments, a vacuum (e.g., a pre-charged vacuum) can be applied to the sheath 112 at any point during retraction of the thrombus extraction device 250. In some embodiments, application of the vacuum can generate instantaneous or nearly instantaneous suction at the distal portion of the sheath 112 that can aspirate any remaining portions of the thrombus 1190 into and/or through the sheath 112. For example, the generated suction can aspirate any of the thrombus 1190 that captured or extruded by the funnel 690. Moreover, in some embodiments, application of a vacuum can facilitate smooth retraction of the captured thrombus 1190 through the sheath 112. For example, a burst of suction generated by application of the vacuum can help inhibit clogging of the sheath 112, and/or help resolve (e.g., break apart) a clog formed in the sheath 112 during retraction.

### V. EXAMPLES

Several aspects of the present technology are set forth in the following examples:

1. A coring element for coring a vascular thrombus within a blood vessel of a patient, the coring element comprising:
a unitary structure having—
   a first region adjacent to a proximal portion of the unitary structure, wherein the first region includes a first mouth configured to core the vascular thrombus;
   a second region distal of the first region, wherein the second region is generally tubular and includes a first plurality of interconnected struts;
   a third region distal of the second region, wherein the third region includes a second mouth configured to core the vascular thrombus; and
   a fourth region distal of the third region, wherein the fourth region is generally tubular and includes a second plurality of interconnected struts.

2. The coring element of example 1 wherein the first mouth is radially offset from the second mouth.

3. The coring element of example 1 or example 2 wherein the unitary structure extends along a longitudinal axis, and wherein the first region includes a pair of first curved struts that curve in opposite directions around the longitudinal axis and intersect at a pair of first junctions to define the first mouth.

4. The coring element of any one of examples 1-3 wherein the unitary structure extends along a longitudinal axis, wherein the third region includes (a) a pair of upper curved struts that curve around the longitudinal axis and intersect each other at an upper junction and (b) a pair of lower curved struts that curve around the longitudinal axis and intersect each other at a lower junction, and wherein the lower and upper curved struts define the second mouth.

5. The coring element of example 4 wherein the lower and upper curved struts define (a) a first mouth portion opening in a first direction generally orthogonal to the longitudinal axis and (b) a second mouth portion opening in a second direction generally orthogonal to the longitudinal axis, and wherein the first and second mouth portions define the second mouth.

6. The coring element of example 5 wherein the first direction is generally opposite to the second direction.

7. The coring element of any one of examples 1-6 wherein the coring element is expandable from a compressed delivery configuration to an expanded deployed configuration.

8. The coring element of example 7 wherein the coring element is configured to self-expand.

9. The coring element of example 8 wherein the coring element is made from a shape memory material.

10. The coring element of any one of examples 1-9 wherein the fourth region of the unitary structure is configured to be connected to a braided filament mesh structure.

11. A dilator assembly for deploying an expandable funnel coupled to a distal portion of an introducer sheath, the dilator assembly comprising:
a first shaft defining a lumen;
a second shaft slidably positioned within the lumen of the first shaft;
a retention sheath coupled to the second shaft and configured to receive and constrain the funnel therein; and
a control assembly including an actuator operably coupled to the first and second shafts, wherein movement of the actuator from a first position to a second position advances the first and second shafts together to deploy the funnel from the retention sheath, and wherein movement of the actuator from the second position to a third position advances the first shaft relative to the second shaft.

12. The dilator assembly of example 11 wherein the retention sheath has substantially a same outer diameter as the first shaft.

13. The dilator assembly of example 11 or example 13 wherein movement of the actuator from the second position to the third position brings a distal portion of the first shaft into contact with a proximal portion of the retention sheath.

14. The dilator assembly of any one of examples 11-13 wherein the control assembly includes—
a housing;
a first shaft hub slidably positioned within the housing and coupled to the first shaft; and
a second shaft hub slidably positioned within the housing and coupled to the second shaft.

15. The dilator assembly of example 14 wherein the first shaft hub is configured to engage the second shaft hub when the actuator is moved from the first position to the second position such that the first and second shafts advance together.

16. The dilator assembly of example 14 or example 15 wherein the first shaft hub is configured to disengage the second shaft hub when the actuator is moved from the second position to the third position such that first shaft advances relative to the second shaft.

17. The dilator assembly of any one of examples 14-16 wherein the first shaft hub is configured to engage the second shaft hub when the actuator is moved from the first position to the second position such that the first and second shafts advance together, and wherein the first shaft hub is configured to disengage the second shaft hub when the actuator is moved from the second position to the third position such that first shaft advances relative to the second shaft.

18. The dilator of assembly of any one of examples 14-17 wherein the second shaft hub includes a first engagement feature, wherein the housing includes a second engagement feature, and wherein the first engagement feature is configured to engage the second engagement feature at the second position to prevent movement of the second shaft hub when the actuator is moved from the second position to the third position.

19. The dilator assembly of example 18 wherein the first engagement feature is a snap feature, and wherein the second engagement feature is a detent formed in the housing.

20. The dilator assembly of any one of examples 14-19, further comprising a biasing member operably coupled to the first shaft hub, wherein the biasing member is configured to bias the first shaft hub from the third position toward the second position.

21. The dilator assembly of any one of examples 11-20 wherein the control assembly further includes a housing, wherein the actuator is movable relative to the housing, wherein the movement of the actuator from the first position to the second position is distal movement of the actuator relative to the housing, and wherein the movement of the actuator from the second position to the third position is further distal movement of the actuator relative to the housing.

22. The dilator assembly of any one of examples 11-21, further comprising the introducer sheath and the funnel.

23. A system for capturing a vascular thrombus within a blood vessel of a patient, the system comprising:
an introducer sheath having a distal portion;
an expandable funnel coupled to the distal portion of the introducer sheath;
a dilator assembly configured to be inserted through the introducer sheath and to deploy the expandable funnel, wherein the dilator assembly includes—
   a first shaft defining a lumen;
   a second shaft slidably positioned within the lumen of the first shaft;
   a retention sheath coupled to the second shaft and configured to receive and constrain the funnel therein; and
   a control assembly including an actuator operably coupled to the first and second shafts, wherein movement of the actuator from a first position to a second position distally advances the first and second shafts together to deploy the funnel from the retention sheath, and wherein movement of the actuator from the second position to a third position advances the first shaft relative to the second shaft; and
a clot removal device configured to be inserted through the introducer sheath to capture at least a portion of the vascular thrombus.

24. The system of example 23 wherein the clot removal device includes an expandable coring element coupled to an expandable capture element, wherein the coring element is configured to separate at least a portion of the vascular thrombus from a wall of the blood vessel, and wherein the capture element is configured to capture and retain the portion of the vascular thrombus separated from the wall of the blood vessel.

25. The system of example 23 or example 24 wherein the funnel has a first length when deployed from the retention sheath, and wherein the coring element has a second length when expanded that is less than the first length.

26. A system for capturing a vascular thrombus within a blood vessel of a patient, the system comprising:
an introducer sheath having a distal portion;
an expandable funnel coupled to the distal portion of the introducer sheath;
a dilator assembly configured to be inserted through the introducer sheath and to deploy the expandable funnel; and
a clot removal device configured to be inserted through the introducer sheath, wherein the clot removal device includes an expandable coring element coupled to an expandable capture element, wherein the coring element includes a first region including a first mouth and a second region including a second mouth, wherein the first and second mouths are configured to separate at least a portion of the vascular thrombus from a wall of the blood vessel, and wherein the capture element is configured to capture and retain the portion of the vascular thrombus separated from the wall of the blood vessel.

27. The system of example 26 wherein the first mouth is radially offset from the second mouth.

28. The system of example 27 wherein the coring element is formed from a unitary structure including a plurality of struts, wherein the struts define the first and second mouths, wherein the struts further define a plurality of interstices, and wherein the first and second mouths are larger than each of the interstices.

### VI. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A coring element for coring a vascular thrombus within a blood vessel of a patient, the coring element comprising:
a unitary structure having—
a first region adjacent to a proximal portion of the unitary structure, wherein the first region includes a first mouth configured to core the vascular thrombus;
a second region distal of the first region, wherein the second region is generally tubular and includes a first plurality of interconnected struts;
a third region distal of the second region, wherein the third region includes a second mouth configured to core the vascular thrombus; and
a fourth region distal of the third region, wherein the fourth region is generally tubular and includes a second plurality of interconnected struts.

2. The coring element of claim 1 wherein the first mouth is radially offset from the second mouth.

3. The coring element of claim 1 wherein the unitary structure extends along a longitudinal axis, and wherein the first region includes a pair of first curved struts that curve in opposite directions around the longitudinal axis and intersect at a pair of first junctions to define the first mouth.

4. The coring element of claim 1 wherein the unitary structure extends along a longitudinal axis, wherein the third region includes (a) a pair of upper curved struts that curve around the longitudinal axis and intersect each other at an upper junction and (b) a pair of lower curved struts that curve around the longitudinal axis and intersect each other at a lower junction, and wherein the lower and upper curved struts define the second mouth.

5. The coring element of claim 4 wherein the lower and upper curved struts define (a) a first mouth portion opening in a first direction generally orthogonal to the longitudinal axis and (b) a second mouth portion opening in a second direction generally orthogonal to the longitudinal axis, and wherein the first and second mouth portions define the second mouth.

6. The coring element of claim 5 wherein the first direction is generally opposite to the second direction.

7. The coring element of claim 1 wherein the coring element is expandable from a compressed delivery configuration to an expanded deployed configuration.

8. The coring element of claim 7 wherein the coring element is configured to self-expand.

9. The coring element of claim 8 wherein the coring element is made from a shape memory material.

10. The coring element of claim 1 wherein the fourth region of the unitary structure is configured to be connected to a braided filament mesh structure.

11. A dilator assembly for deploying an expandable funnel coupled to a distal portion of an introducer sheath, the dilator assembly comprising:
a first shaft defining a lumen;
a second shaft slidably positioned within the lumen of the first shaft;
a retention sheath coupled to the second shaft and configured to receive and constrain the funnel therein; and
a control assembly including an actuator operably coupled to the first and second shafts, wherein movement of the actuator from a first position to a second position advances the first and second shafts together to deploy the funnel from the retention sheath, and wherein movement of the actuator from the second position to a third position advances the first shaft relative to the second shaft.

12. The dilator assembly of claim 11 wherein the retention sheath has substantially a same outer diameter as the first shaft.

13. The dilator assembly of claim 11 wherein movement of the actuator from the second position to the third position brings a distal portion of the first shaft into contact with a proximal portion of the retention sheath.

14. The dilator assembly of claim 11 wherein the control assembly includes—
a housing;
a first shaft hub slidably positioned within the housing and coupled to the first shaft; and
a second shaft hub slidably positioned within the housing and coupled to the second shaft.

15. The dilator assembly of claim 14 wherein the first shaft hub is configured to engage the second shaft hub when the actuator is moved from the first position to the second position such that the first and second shafts advance together.

16. The dilator assembly of claim 14 wherein the first shaft hub is configured to disengage the second shaft hub when the actuator is moved from the second position to the third position such that first shaft advances relative to the second shaft.

17. The dilator assembly of claim 14 wherein the first shaft hub is configured to engage the second shaft hub when the actuator is moved from the first position to the second position such that the first and second shafts advance together, and wherein the first shaft hub is configured to disengage the second shaft hub when the actuator is moved from the second position to the third position such that first shaft advances relative to the second shaft.

18. The dilator of assembly of claim 14 wherein the second shaft hub includes a first engagement feature, wherein the housing includes a second engagement feature, and wherein the first engagement feature is configured to engage the second engagement feature at the second position to prevent movement of the second shaft hub when the actuator is moved from the second position to the third position.

19. The dilator assembly of claim 18 wherein the first engagement feature is a snap feature, and wherein the second engagement feature is a detent formed in the housing.

20. The dilator assembly of claim 14, further comprising a biasing member operably coupled to the first shaft hub, wherein the biasing member is configured to bias the first shaft hub from the third position toward the second position.

21. The dilator assembly of claim 11 wherein the control assembly further includes a housing, wherein the actuator is movable relative to the housing, wherein the movement of the actuator from the first position to the second position is distal movement of the actuator relative to the housing, and wherein the movement of the actuator from the second position to the third position is further distal movement of the actuator relative to the housing.

22. The dilator assembly of claim 11, further comprising the introducer sheath and the funnel.

23. A system for capturing a vascular thrombus within a blood vessel of a patient, the system comprising:
an introducer sheath having a distal portion;
an expandable funnel coupled to the distal portion of the introducer sheath;
a dilator assembly configured to be inserted through the introducer sheath and to deploy the expandable funnel, wherein the dilator assembly includes—
a first shaft defining a lumen;
a second shaft slidably positioned within the lumen of the first shaft;
a retention sheath coupled to the second shaft and configured to receive and constrain the funnel therein; and
a control assembly including an actuator operably coupled to the first and second shafts, wherein movement of the actuator from a first position to a second position distally advances the first and second shafts together to deploy the funnel from the retention sheath, and wherein movement of the actuator from the second position to a third position advances the first shaft relative to the second shaft; and
a clot removal device configured to be inserted through the introducer sheath to capture at least a portion of the vascular thrombus.

24. The system of claim 23 wherein the clot removal device includes an expandable coring element coupled to an expandable capture element, wherein the coring element is configured to separate at least a portion of the vascular thrombus from a wall of the blood vessel, and wherein the capture element is configured to capture and retain the portion of the vascular thrombus separated from the wall of the blood vessel.

25. The system of claim 23 wherein the funnel has a first length when deployed from the retention sheath, and wherein the coring element has a second length when expanded that is less than the first length.

26. A system for capturing a vascular thrombus within a blood vessel of a patient, the system comprising:
an introducer sheath having a distal portion;
an expandable funnel coupled to the distal portion of the introducer sheath;
a dilator assembly configured to be inserted through the introducer sheath and to deploy the expandable funnel; and
a clot removal device configured to be inserted through the introducer sheath, wherein the clot removal device includes an expandable coring element coupled to an expandable capture element, wherein the coring element includes a first region including a first mouth and a second region including a second mouth, wherein the first and second mouths are configured to separate at least a portion of the vascular thrombus from a wall of the blood vessel, and wherein the capture element is configured to capture and retain the portion of the vascular thrombus separated from the wall of the blood vessel.

27. The system of claim 26 wherein the first mouth is radially offset from the second mouth.

28. The system of claim 27 wherein the coring element is formed from a unitary structure including a plurality of struts, wherein the struts define the first and second mouths, wherein the struts further define a plurality of interstices, and wherein the first and second mouths are larger than each of the interstices.
